(19) United States
(12) Patent Application Publication
(10) Pub. No.: US 2022/0042078 A1
(43) Pub. Date: Feb. 10, 2022

(54) IDENTIFICATION AND ANALYSIS OF MICROBIAL SAMPLES BY RAPID INCUBATION AND NUCLEIC ACID ENRICHMENT

(71) Applicant: Illumina, Inc., San Diego, CA (US)

(72) Inventor: Clifford Lee Wang, Redwood City, CA (US)

(21) Appl. No.: 17/280,746

(22) PCT Filed: Apr. 28, 2020
(86) PCT No.: PCT/US2020/030310

§ 371 Date: Mar. 26, 2021

Related U.S. Application Data
(60) Provisional application No. 62/840,322, filed on Apr. 29, 2019.

(51) Int. Cl. C12Q 1/689; C12Q 1/6806

(52) U.S. Cl. CPC C12Q 1/689; C12Q 1/6806

(57) ABSTRACT

The disclosure relates to methods, compositions, and kits for the identification and analysis of microorganisms in a sample using nucleoside or nucleotide analogs.

### CROSS REFERENCE TO RELATED APPLICATIONS

This application claims priority under 35 U.S.C. § 119 from Provisional Application Ser. No. 62/840,322, filed Apr. 29, 2019, the disclosures of which are incorporated herein by reference.

### TECHNICAL FIELD

The disclosure relates to methods, compositions, and kits for the identification and analysis of microorganisms in a sample using nucleoside or nucleotide analogs.

### BACKGROUND

Determining whether a patient has a microbial infection is a common clinical challenge. Sepsis is the most common cause of death in hospitalized patients, with an estimated 200,000 deaths annually in the USA. However, sepsis is an imprecise clinical syndrome, with a variable clinical presentation. Diagnosis is usually based on suspicion of infection, combined with signs of organ dysfunction. Early diagnosis of sepsis and administration of antibiotics is vital because progression to severe sepsis or septic shock has serious consequences. Unfortunately, differentiating between sepsis and other inflammatory conditions is often challenging in seriously ill patients. Detecting bacterial infections in blood is a key step in the diagnosis of sepsis, and initiating treatment with antimicrobials. However, blood cultures are negative in 60 to 70% of patients with severe sepsis, and >80% were negative in a study. In addition, traditional microbiology methods take too long to influence first line therapy against pathogenic bacteria. Developments in PCR and mass spectrometry have increased the likelihood of identifying bacteria in blood samples, but often rely on time-consuming pre-analytical processing such as blood culture in order to increase pathogen load. Proxies for infection include increased circulating cytokines and acute phase proteins, such as C-reactive protein; although, their concentrations also increase during physiological events such as parturition, or pathological tissue damage such as burns. Typically for sepsis, a blood culture test is done to try to identify what type of bacteria or fungi has caused an infection in the blood. Blood cultures are collected separately from other blood tests and often they are taken more than once from different veins. It can take several days to get the results of a blood culture. Due to the in vitro culture conditions, only a third to a half of people with sepsis will have blood cultures that are positive, meaning that bacteria actually grow and proliferate in the in vitro conditions.

### SUMMARY

Currently, detection of bacteria often requires culturing in order to (1) isolate bacteria for analysis and (2) reduce any contaminating background cells or other material that could make analysis difficult or impossible. For example, in patients with sepsis, blood must be cultured in order to isolate pathogenic bacteria. Similarly, in the monitoring of food, samples must be cultured in order to isolate contaminating microbes. Unfortunately, the standard culturing process can take several days. In the case of sepsis, this lag time can lead to the unnecessary administration of antibiotics or a misdiagnosis, leading to patient complications or death. In the food industry, this lag time delays information that would lead to recalls or other preventive measures. Thus, the rapid detection of active infections can enable measures that can reduce problems and save human lives.

While PCR detection methods may not require extended culturing, PCR, in contrast to unbiased sequencing approaches, requires a priori knowledge of the genome sequence of the organisms of interest. That is, a researcher has to know what they are looking for, and this likely will not be the case for rare or undiscovered organisms. Additionally, PCR-dependent methods only detect the presence of genetic material in the sample and cannot distinguish whether that material came from a live or dead organism. In many cases, the identification of active infections caused by live microorganisms is the most important consideration for treatment options, or even for identification of contaminants in foodstuffs or the environment.

The disclosure provides a method for the identification and analysis of viable and/or proliferating microorganisms in a sample, comprising: (a) obtaining a sample having or suspected of having one or more types of microorganisms; (b) incubating the sample in the presence of one or more types of nucleoside or nucleotide analogs, wherein the one or more types of nucleoside or nucleotide analogs are incorporated into newly synthesized microbial nucleic acids; (c) labelling newly synthesized microbial nucleic acids by contacting the newly synthesized microbial nucleic acids with a labelling reagent that selectively binds to or with the one or more types of nucleoside or nucleotide analogs; (d) isolating or purifying the labelled newly synthesized microbial nucleic acids; and (e) determining the identity of the viable and/or proliferating microorganisms in the sample based upon sequencing or determining the identity of the isolated or purified newly synthesized microbial nucleic acids. In another embodiment, the sample is obtained from a subject suspected of having or having a microbial infection. In yet another embodiment, the subject is suspected of having or has sepsis. In a further embodiment, for (a), the obtained sample is processed using a dehosting method prior to (b) in order to selectively remove non-microbial nucleic acids. In yet a further embodiment, the dehosting method comprises: removing non-microbial nucleic acids by: (i) selectively cleaving non-microbial DNA by contacting the obtained sample with a recombinant protein comprising: a binding domain that selectively binds to non-microbial nucleic acids bound by histone(s) or to non-microbial nucleic acids comprising methylated CpG residues, and a nuclease domain having activity to cleave nucleic acids; or (ii) use of an affinity agent that is bound to a solid substrate that selectively binds to nucleic acids bound by histone(s) or selectively binds to methylated CpG residues of non-microbial nucleic acids. In a certain embodiment, the sample is an environmental sample obtained from an environmental test site. In another embodiment, the environmental site is being tested for microbial contamination. In yet another embodiment, the sample is a sample obtained from a foodstuff suspected of microbial contamination. In a further embodiment, the one or more types of microorganisms are bacteria, fungi, viruses, algae, archaea, and/or protozoa. In yet a further embodiment, the bacteria are selected from Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and/or Yersinia pseudotuberculosis. In another embodiment, the fungi are selected from Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp., Cadophora spp., Candida albicans, Cercospora apii, Chrysosporium spp., Cladosporium spp., Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp., Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp., Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp., Trichophyton spp., Trichosporon spp., and/or Zopfia rosatii. In yet another embodiment, the viruses are selected from Simplexvirus, Varicellovirus, Cytomegalovirus, Roseolovirus, Lympho-cryptovirus, Rhadinovirus, Mastadenovirus, α-Papillomavirus, β-Papillomavirus, X-Papillomavirus, γ-Papillomavirus, Mupapillomavirus, Nupapillomavirus, Alphapolyomavirus, Betapolyomavirus, γ-Polyomavirus, Deltapolyomavirus, Molluscipoxvirus, Orthopoxvirus, Parapoxvirus, α-Torquevirus, β-Torquevirus, γ-Torquevirus, Cyclovirus, Gemycircular, Gemykibivirus, Gemyvongvirus, Erythrovirus, Dependovirus, Bocavirus, Orthohepadnavirus, Gammaretrovirus, Deltaretrovirus, Lentivirus, Simiispumavirus, Coltivirus, Rotavirus, Seadornavirus, α-Coronavirus, β-Coronavirus, Torovirus, Mamastrovirus, Norovirus, Sapovirus, Flavivirus, Hepacivirus, Pegivirus, Orthohepevirus, Cardiovirus, Cosavirus, Enterovirus, Hepatovirus, Kobuvirus, Parechovirus, Rosavirus, Salivirus, Alphavirus, Rubivirus, Ebolavirus, Marburgvirus, Henipavirus, Morbilivirus, Respirovirus, Rubulavirus, Metapneumovirus, Orthopneumovirus, Ledantevirus, Lyssavirus, Vesiculovirus, Mammarenavirus, Orthohantavirus, Orthonairovirus, Orthobunyavirus, Phlebovirus, α-Influenzavirus, β-Influenzavirus, γ-Influenzavirus, Quaranjavirus, Thogotovirus, and/or Deltavirus. In a further embodiment, the one or more types of nucleoside or nucleotide analogs are selected from 2-ethynyl-adenosine, N6-propargyl-adenosine, 2′-(O-propargyl)-adenosine, 3′-(O-propargyl)-adenosine, 5-ethynyl-cytidine, 5-ethynyl-2′-deoxycytidine, 2′-(O-propargyl)-cytidine, 3′-(O-propargyl)-cytidine, 2′-(O-propargyl)-guanosine, 3′-(O-propargyl)-guanosine, 5-ethynyl-uridine, 5-ethynyl-2′-deoxyuridine, 2′-(O-propargyl)-uridine, 3′-(O-propargyl)-uridine, (2'S)-2′-deoxy-2′-fluoro-5-ethynyluridine, (2'S)-2′-fluoro-5-ethynyluridine, 2′ (S)-2′-deoxy-2′-fluoro-5-ethynyluridine, (2'S)-2′-fluoro-5-ethynyluridine, 8-azido-adenosine, N⁶-(6-azido)hexyl-2′deoxy-adenosine, 2′-azido-2′-deoxyadenosine, 5-azidomethyl-uridine, 5-(15-azido-4,7,10,13-tetraoxa-pentadecanoyl-aminoallyl)-2′-deoxyuridine, 5-(3-azidopropyl)-uridine, 5-azido-PEG₄-uridine, 5-azido-PEG₄-cytidine, 5-azido-PEG₄-2′-deoxycytidine, 5-bromo-2′deoxyuridine, 5-bromouridine, 5-iodo-2′deoxyuridine, and 5-iodouridine. In yet a further embodiment, the one or more types of nucleoside or nucleotide analogs are selected from 2-ethynyl-adenosine, N6-propargyl-adenosine, 2′-(O-propargyl)-adenosine, 3′-(O-propargyl)-adenosine, 5-ethynyl-cytidine, 5-ethynyl-2′-deoxycytidine, 2′-(O-propargyl)-cytidine, 3′-(O-propargyl)-cytidine, 2′-(O-propargyl)-guanosine, 3′-(O-propargyl)-guanosine, 5-ethynyl-uridine, 5-ethynyl-2′-deoxyuridine, 2′-(O-propargyl)-uridine, 3′-(O-propargyl)-uridine, (2'S)-2′-deoxy-2′-fluoro-5-ethynyluridine, (2'S)-2′-fluoro-5-ethynyluridine, 2′ (S)-2′-deoxy-2′-fluoro-5-ethynyluridine, and (2'S)-2′-fluoro-5-ethynyluridine. In yet another embodiment, the one or more types of nucleoside or nucleotide analogs are selected from 8-azido-adenosine, N⁶-(6-azido)hexyl-2′deoxy-adenosine, wherein the one or more types of nucleoside or nucleotide analogs are selected from 2′-azido-2′-deoxyadenosine, 5-azidomethyl-uridine, 5-(15-azido-4,7,10,13-tetraoxa-pentadecanoyl-aminoallyl)-2′-deoxyuridine, 5-(3-azidopropyl)-uridine, 5-azido-PEG₄-uridine, 5-azido-PEG₄-cytidine, and 5-azido-PEG₄-2′-deoxycytidine. In a further embodiment, the one or more types of nucleoside or nucleotide analogs are selected from 5-bromo-2′deoxyuridine, 5-bromouridine, 5-iodo-2′deoxyuridine, and 5-iodouridine. In yet a further embodiment, the sample is incubated in the presence of one or more types of nucleoside or nucleotide analogs for 5 min to 180 min. In another embodiment, the sample is incubated in the presence of one or more types of nucleoside or nucleotide analogs for 30 min to 120 min. In yet another embodiment, the labeling reagent is an antibody that binds with high specificity to the one or more types of nucleoside or nucleotide analogs. In a particular embodiment, the antibody binds with high specificity to 5-bromo-2′deoxyuridine, or iododeoxyuridine. In another embodiment, the labelling reagent binds to or with the one or more types of nucleoside or nucleotide analogs via click chemistry, a strained [3+2] cycloaddition reaction, or a Staudinger ligation. In yet another embodiment, the labelling reagent comprises an azide group which binds to nucleoside or nucleotide analogs comprising an alkynyl group via click chemistry. In a further embodiment, the labelling reagent comprises an alkynyl group which binds to nucleoside or nucleotide analogs comprising an azide group via click chemistry. In a certain embodiment, the labelling reagent comprises a biotin group. In a further embodiment, the labelling reagent comprising a biotin group is selected from:

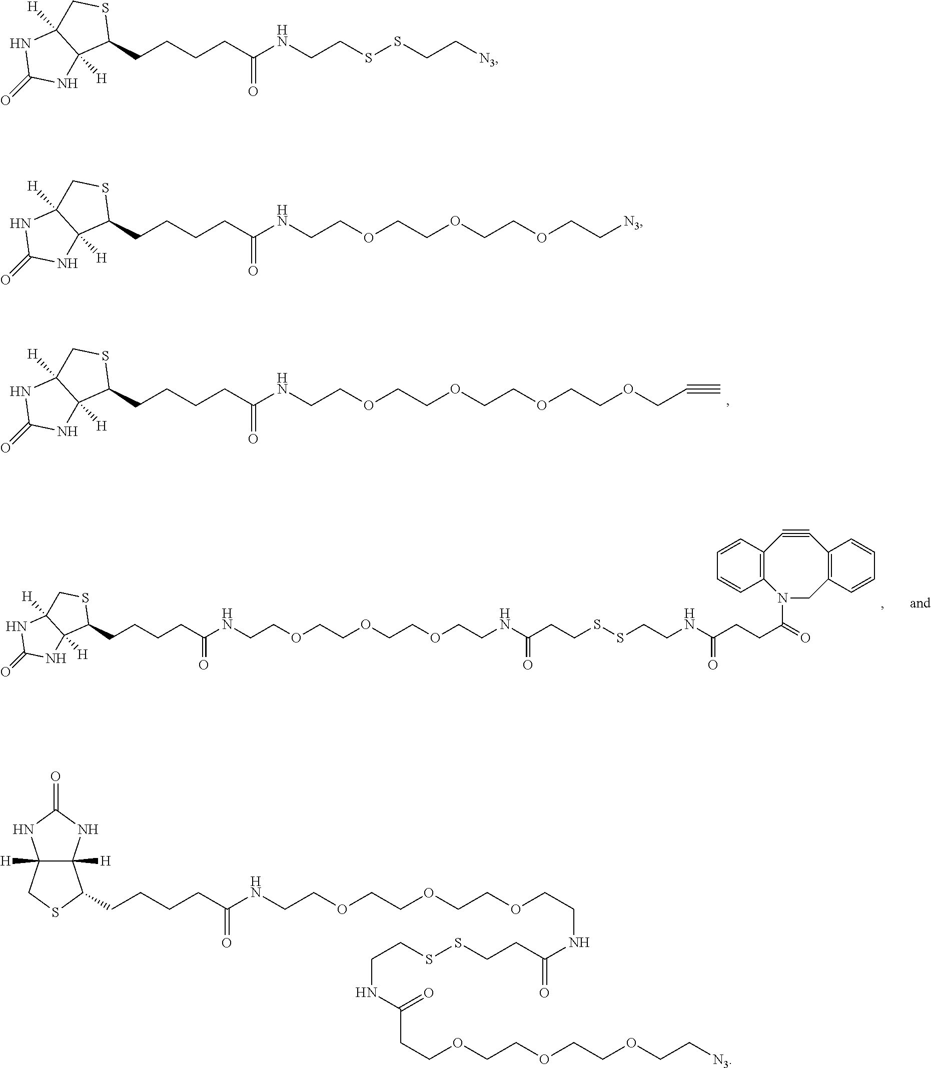 [SMILES: [N-]=[N+]=NCCSSCCNC(=O)CCCC[C@@H]1SC[C@@H]2NC(=O)N[C@@H]21 ; [N-]=[N+]=NCCOCCOCCOCCNC(=O)CCCC[C@@H]1SC[C@@H]2NC(=O)N[C@@H]21 ; C#CCOCCOCCOCCOCCNC(=O)CCCC[C@@H]1SC[C@@H]2NC(=O)N[C@@H]21 ; O=C(CCCC[C@@H]1SC[C@@H]2NC(=O)N[C@@H]21)NCCOCCOCCOCCNC(=O)CCSSCCNC(=O)CCC(=O)N1Cc2ccccc2C#Cc2ccccc21 ; [N-]=[N+]=NCCOCCOCCOCCC(=O)NCCSSCCC(=O)NCCOCCOCCOCCNC(=O)CCCC[C@@H]1SC[C@@H]2NC(=O)N[C@@H]21]

In a further embodiment, the labelling reagent further comprises a chemically cleavable linker or enzymatically cleavable linker. In yet a further embodiment, the cleavable linker is an acid-labile-based linker or a disulfide-based linker. In a certain embodiment, the acid-labile-based linker comprises hydrazone or cis-aconityl groups. In another embodiment, the enzymatically cleavable linker comprises a peptide-based linker or a β-glucuronide-based linker. In yet another embodiment, a pulldown agent is used to isolate or purified the labelled newly synthesized microbial nucleic acids. In a further embodiment, the pulldown reagent is an antibody immobilized onto a solid support, wherein the antibody binds with high specificity to labelling reagent, or with high specificity to the one or more types of nucleoside or nucleotide analogs. In yet a further embodiment, the pulldown reagent is streptavidin or avidin immobilized onto a solid support, and wherein the labelling reagent comprises a biotin group. In a certain embodiment, the solid support is nano- or micro-materials, beads or a plate. In another embodiment, the labelling reagent or label is removed or cleaved from the isolated or purified newly synthesized microbial nucleic acids prior (e) described above. In yet another embodiment, the identity of the isolated or purified newly synthesized microbial nucleic acids is determined by using a microarray comprising probes to nucleic acids from different microorganisms. In a further embodiment, the identity of the isolated or purified newly synthesized microbial nucleic acids is determined by: (i) amplifying the isolated or purified newly synthesized microbial nucleic acids using a first PCR based method using primers containing a fluorescent dye to form labelled products, wherein the primers comprise a sequence that is specific to a conserved microbial 16S rRNA gene region; (ii) applying the labelled products to a microarray comprising probes that comprise unique 16s rRNA variable region sequences from 20 or more microorganisms; (iii) determining the identity of the viable and/or proliferating microorganisms based upon imaging the microarray for fluorescent hybridization products and determining the identity of the microorganism based upon the sequence of the microarray probe. In another embodiment, the identity of the isolated or purified newly synthesized microbial nucleic acids is determined or confirmed by sequencing the isolated or purified newly synthesized microbial nucleic acids. In yet another embodiment, the isolated or purified newly synthesized microbial nucleic acids are sequenced using a transposome-based sequencing method. In a further embodiment, sequencing of the newly synthesized microbial nucleic acids is by: (a) applying the isolated or purified newly synthesized microbial nucleic acids to bead-linked transposomes, wherein the bead-linked transposomes mediate the simultaneous fragmentation of microbial nucleic acids and the addition of sequencing primers; (b) amplifying the microbial nucleic acid fragments with primers that comprise index and adapter sequences to form library of amplified products; (c) washing and pooling the library of amplified products; (d) sequencing the library of amplified products; and (e) determining the identity of the viable and/or proliferating microorganisms based upon correlating the sequences obtained from the library of amplified products with databases of known sequences of microorganisms using bioinformatic analysis. In another embodiment, the newly synthesized microbial nucleic acids are RNA, wherein the microbial RNA is reversed transcribed into cDNA prior (e) described above, and wherein the gene expression of the viable and/or proliferating microorganisms can be determined based on analyzing the expression level of gene products from newly synthesized microbial RNA using a microarray and/or by sequencing.

In a particular embodiment, the disclosure also provides a method for determining the effectiveness of an antimicrobial agent in modulating the growth and proliferation of microorganism(s) in a sample, comprising: (a) obtaining a sample having or suspected of having one or more types of microorganisms; (b) splitting the sample into two samples, a control sample and a treated sample; (c) incubating the control sample in the presence of one or more types of nucleoside or nucleotide analogs, wherein the one or more types of nucleoside or nucleotide analogs are incorporated into newly synthesized microbial nucleic acids; (c′) incubating the treated sample in the presence of one or more types of nucleoside or nucleotide analogs and an antimicrobial agent, wherein the one or more types of nucleoside or nucleotide analogs are incorporated into newly synthesized microbial nucleic acids; (d) labelling newly synthesized microbial nucleic acids of the control sample and the treated sample by contacting the newly synthesized microbial nucleic acids with a labelling reagent that selectively binds to or with the one or more types of nucleoside or nucleotide analogs; (e) isolating or purifying the labelled newly synthesized microbial nucleic acids from the control sample and the treated sample; (f) determining the gene expression level, and/or amounts or identity of the isolated or purified newly synthesized microbial nucleic acids in the control sample; (f′) determining the gene expression level, and/or amounts and identity of the isolated or purified newly synthesized microbial nucleic acids in the treated sample; (g) comparing and determining any changes in the gene expression level and/or amounts and/or identity of the isolated or purified newly synthesized microbial nucleic acids in the control sample with the gene expression level and/or amounts or identity of the isolated or purified newly synthesized microbial nucleic acids in the treated sample, wherein if there is a decrease in the gene expression level of the newly synthesized microbial nucleic acids in the treated sample v. the control sample, or there is decrease in the amounts and/or identity of the newly synthesized microbial nucleic acids in the treated sample v. the control sample indicates that the antimicrobial agent is effective in modulating the growth and proliferation of the microorganism(s). In another embodiment, the antimicrobial agent is selected from an antibiotic, an antifungal, and an antiviral. In a further embodiment, the antibiotic is selected from amoxicillin, ampicillin, bacampicillin, carbenicillin, cloxacillin, dicloxacillin, flucloxacillin, mezlocillin, nafcillin, oxacillin, penicillin G, penicillin V, piperacillin, pivampicillin, pivmecillinam, ticarcillin, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cefcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, cefoperazone, ceftazidime, cefclidine, cefepime, cefluprenam, cefoselis, cefozopran, cefpirome, cefquinome, ceftobiprole, ceftaroline, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, cefuracetime, ceftioxide, aztreonam, imipenem, doripenem, ertapenem, meropenem, azithromycin, erythromycin, clarithromycin, dirithromycin, roxithromycin, telithromycin, clindamycin, lincomycin, amikacin, gentamicin, kanamycin, neomycin, netilmicin, paromomycin, streptomycin, tobramycin, flumequine, nalidixic acid, oxolinic acid, piromidic acid, pipemidic acid, rosoxacin, ciprofloxacin, enoxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, besifloxacin, delafloxacin, clinafloxacin, gemifloxacin, prulifloxacin, sitafloxacin, trovafloxacin, sulfamethizole, sulfamethoxazole, sulfisoxazole, trimethoprim-sulfamethoxazole, demeclocycline, doxycycline, minocycline, oxytetracycline, tetracycline, tigecycline, vancomycin, teicoplanin, telavancin, linezolid, cycloserine, rifampin, rifabutin, rifapentine, rifalazil, viomycin, capreomycin, bacitracin, polymyxin B, chloramphenicol, metronidazole, tinidazole, and nitrofurantoin. In yet a further embodiment, the antifungal is selected from amorolfine, butenafine, naftifine, terbinafine, bifonazole, butoconazole, clotrimazole, econazole, fenticonazole, ketoconazole, isoconazole, luliconazole, miconazole, omoconazole, oxiconazole, sertaconazole, sulconazole, tioconazole, terconazole, albaconazole, efinaconazole, fluconazole, isavuconazole, itraconazole, posaconazole, ravuconazole, terconazole, voriconazole, abafungin, amphotericin B, nystatin, natamycin, trichomycin, anidulafungin, caspofungin, micafungin, tolnaftate, flucytosine, butenafine, griseofulvin, ciclopirox, selenium sulfide, tavaborole. In another embodiment, the antiviral is selected from acyclovir, brivudine, docosanol, famciclovir, foscarnet, idoxuridine, penciclovir, trifluridine, vidarabine, cytarabine, valacyclovir, tromatandine, pritelivir, amantadine, rimantadine, oseltamivir, peramivir, zanamivir, asunaprevir, boceprevir, ciluprevir, danoprevir, faldaprevir, glecaprevir, grazoprevir, narlaprevir, paritaprevir, simeprevir, sovaprevir, telaprevir, vaniprevir, vedroprevir, voxilaprevir, daclatasvir, elbasvir, ledipasvir, odalasvir, ombitasvir, pibrentasvir, ravidasvir, ruzasvir, samatasvir, velpatasvir, beclabuvir, dasabuvir, deleobuvir, filibuvir, setrobuvir, sofosbuvir, radalbuvir, uprifosbuvir, lamivudine, telbivudine, clevudine, adefovir, tenofvir disoproxil, tenofovir alafenamide, enfuvirtide, maraviroc, vicriviroc, cenicriviroc, PRO 140, ibalizumab, fostemsavir, didanosine, emtricitabine, lamivudine, stavudine, zidovudine, amdoxovir, apricitabine, censavudine, elvucitabine, racivir, stampidine, 4′-ethynyl-2-fluoro-2′-deoxyadenosine, zalcitabine, efavirenz, nevirapine, delavirdine, etravirine, rilpivirine, doravirine, dolutegravir, elvitegravir, raltegravir, BI 224436, cabotegravir, bictegravir, MK-2048, bevirimat, BMS-955176, amprenavir, fosamprenavir, indinavir, lopinavir, nelfinavir, ritonavir, saquinavir, atazanavir, darunavir, tipranavir, dolutegravir, elvitegravir, raltegravir, BI 224436, cabotegravir, bictegravir, MK-2048, cobicistat, ritonavir, interferon-α, peginterferon-α, methisazone, rifampicin, imiquimod, resiquimod, podophyllotoxin, fomivirsen, cidofovir, pleconaril, favipiravir, galidesivir, remdesivir, mericitabine, MK-608, NITD008, moroxydine, tromantadine, and triazavirin. In yet a further embodiment, the sample is obtained from a subject suspected of having or having a microbial infection. In a particular embodiment, the subject is suspected of having or has sepsis. In another embodiment, the one or more types of microorganisms are bacteria, fungi, and/or viruses. In yet another embodiment, the bacteria are selected from Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and/or Yersinia pseudotuberculosis. In a further embodiment, the fungi are selected from Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp., Blastomyces spp., Cadophora spp., Candida albicans, Cercospora apii, Chrysosporium spp., Cladosporium spp., Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp., Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp., Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp., Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp., Trichophyton spp., Trichosporon spp., and/or Zopfia rosatii. In yet a further embodiment, the viruses are selected from Simplexvirus, Varicellovirus, Cytomegalovirus, Roseolovirus, Lympho-cryptovirus, Rhadinovirus, Mastadenovirus, α-Papillomavirus, β-Papillomavirus, X-Papillomavirus, γ-Papillomavirus, Mupapillomavirus, Nupapillomavirus, Alphapolyomavirus, Betapolyomavirus, γ-Polyomavirus, Deltapolyomavirus, Molluscipoxvirus, Orthopoxvirus, Parapoxvirus, α-Torquevirus, β-Torquevirus, γ-Torquevirus, Cyclovirus, Gemycircular, Gemykibivirus, Gemyvongvirus, Erythrovirus, Dependovirus, Bocavirus, Orthohepadnavirus, Gammaretrovirus, Deltaretrovirus, Lentivirus, Simiispumavirus, Coltivirus, Rotavirus, Seadornavirus, α-Coronavirus, β-Coronavirus, Torovirus, Mamastrovirus, Norovirus, Sapovirus, Flavivirus, Hepacivirus, Pegivirus, Orthohepevirus, Cardiovirus, Cosavirus, Enterovirus, Hepatovirus, Kobuvirus, Parechovirus, Rosavirus, Salivirus, Alphavirus, Rubivirus, Ebolavirus, Marburgvirus, Henipavirus, Morbilivirus, Respirovirus, Rubulavirus, Metapneumovirus, Orthopneumovirus, Ledantevirus, Lyssavirus, Vesiculovirus, Mammarenavirus, Orthohantavirus, Orthonairovirus, Orthobunyavirus, Phlebovirus, α-Influenzavirus, β-Influenzavirus, γ-Influenzavirus, Quaranjavirus, Thogotovirus, and/or Deltavirus. In a certain embodiment, the one or more types of nucleoside or nucleotide analogs are selected from 2-ethynyl-adenosine, N6-propargyl-adenosine, 2′-(O-propargyl)-adenosine, 3′-(O-propargyl)-adenosine, 5-ethynyl-cytidine, 5-ethynyl-2′-deoxycytidine, 2′-(O-propargyl)-cytidine, 3′-(O-propargyl)-cytidine, 2′-(O-propargyl)-guanosine, 3′-(O-propargyl)-guanosine, 5-ethynyl-uridine, 5-ethynyl-2′-deoxyuridine, 2′-(O-propargyl)-uridine, 3′-(O-propargyl)-uridine, (2'S)-2′-deoxy-2′-fluoro-5-ethynyluridine, (2'S)-2′-fluoro-5-ethynyluridine, 2′ (S)-2′-deoxy-2′-fluoro-5-ethynyluridine, (2'S)-2′-fluoro-5-ethynyluridine, 8-azido-adenosine, N⁶-(6-azido)hexyl-2′deoxy-adenosine, 2′-azido-2′-deoxyadenosine, 5-azidomethyl-uridine, 5-(15-azido-4,7,10,13-tetraoxa-pentadecanoyl-aminoallyl)-2′-deoxyuridine, 5-(3-azidopropyl)-uridine, 5-azido-PEG₄-uridine, 5-azido-PEG₄-cytidine, 5-azido-PEG₄-2′-deoxycytidine, 5-bromo-2′deoxyuridine, 5-bromouridine, 5-iodo-2′deoxyuridine, and 5-iodouridine. In another embodiment, the one or more types of nucleoside or nucleotide analogs are selected from 2-ethynyl-adenosine, N6-propargyl-adenosine, 2′-(O-propargyl)-adenosine, 3′-(O-propargyl)-adenosine, 5-ethynyl-cytidine, 5-ethynyl-2′-deoxycytidine, 2′-(O-propargyl)-cytidine, 3′-(O-propargyl)-cytidine, 2′-(O-propargyl)-guanosine, 3′-(O-propargyl)-guanosine, 5-ethynyl-uridine, 5-ethynyl-2′-deoxyuridine, 2′-(O-propargyl)-uridine, 3′-(O-propargyl)-uridine, (2'S)-2′-deoxy-2′-fluoro-5-ethynyluridine, (2'S)-2′-fluoro-5-ethynyluridine, 2′ (S)-2′-deoxy-2′-fluoro-5-ethynyluridine, and (2'S)-2′-fluoro-5-ethynyluridine. In yet another embodiment, the one or more types of nucleoside or nucleotide analogs are selected from 8-azido-adenosine, N⁶-(6-azido)hexyl-2′deoxy-adenosine, wherein the one or more types of nucleoside or nucleotide analogs are selected from 2′-azido-2′-deoxyadenosine, 5-azidomethyl-uridine, 5-(15-azido-4,7,10,13-tetraoxa-pentadecanoyl-aminoallyl)-2′-deoxyuridine, 5-(3-azidopropyl)-uridine, 5-azido-PEG₄-uridine, 5-azido-PEG₄-cytidine, and 5-azido-PEG₄-2′-deoxycytidine. In a particular embodiment, the one or more types of nucleoside or nucleotide analogs are selected from 5-bromo-2′deoxyuridine, 5-bromouridine, 5-iodo-2′deoxyuridine, and 5-iodouridine. In another embodiment, the control sample and the treated sample are both incubated for the same period time in the presence of one or more types of nucleoside or nucleotide analogs for 5 min to 180 min. In yet another embodiment, the control sample and the treated sample are both incubated for the same period time in the presence of one or more types of nucleoside or nucleotide analogs for 30 min to 120 min. In a further embodiment, the labeling reagent is an antibody that binds with high specificity to the one or more types of nucleoside or nucleotide analogs. In yet a further embodiment, the antibody binds with high specificity to 5-bromo-2′deoxyuridine, or iododeoxyuridine. In a certain embodiment, the labelling reagent binds to or with the one or more types of nucleoside or nucleotide analogs via click chemistry, a strained [3+2] cycloaddition reaction, or a Staudinger ligation. In another embodiment, the labelling reagent comprises an azide group which binds to nucleoside or nucleotide analogs comprising an alkynyl group via click chemistry. In yet another embodiment, the labelling reagent comprises an alkynyl group which binds to nucleoside or nucleotide analogs comprising an azide group via click chemistry. In a further embodiment, the labelling reagent comprises a biotin group. In a particular embodiment, the labelling reagent comprising a biotin group is selected from:

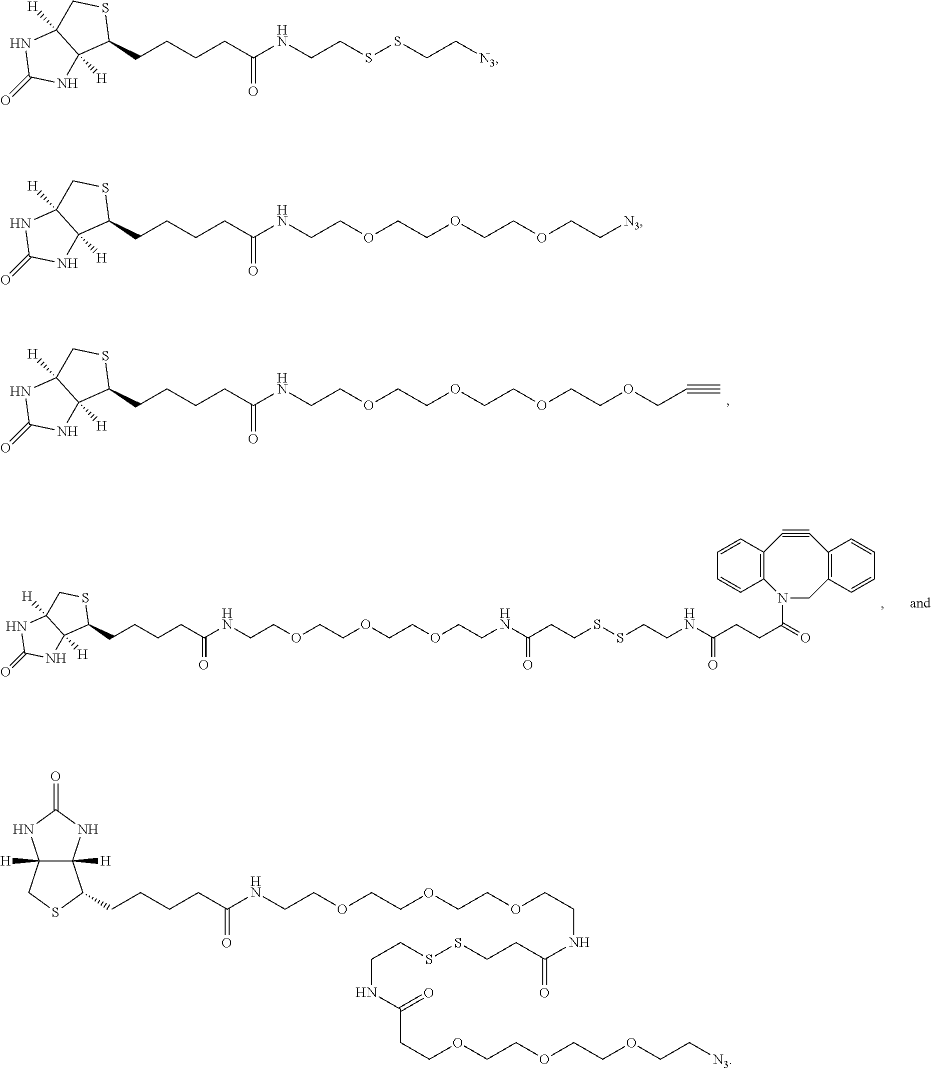 [SMILES: [N-]=[N+]=NCCSSCCNC(=O)CCCC[C@@H]1SC[C@@H]2NC(=O)N[C@@H]21 ; [N-]=[N+]=NCCOCCOCCOCCNC(=O)CCCC[C@@H]1SC[C@@H]2NC(=O)N[C@@H]21 ; C#CCOCCOCCOCCOCCNC(=O)CCCC[C@@H]1SC[C@@H]2NC(=O)N[C@@H]21 ; O=C(CCCC[C@@H]1SC[C@@H]2NC(=O)N[C@@H]21)NCCOCCOCCOCCNC(=O)CCSSCCNC(=O)CCC(=O)N1Cc2ccccc2C#Cc2ccccc21 ; [N-]=[N+]=NCCOCCOCCOCCC(=O)NCCSSCCC(=O)NCCOCCOCCOCCNC(=O)CCCC[C@@H]1SC[C@@H]2NC(=O)N[C@@H]21]

In another embodiment, the labelling reagent further comprises a chemically cleavable linker or enzymatically cleavable linker. In yet another embodiment, cleavable linker is an acid-labile-based linker or a disulfide-based linker. In a further embodiment, the acid-labile-based linker comprises hydrazone or cis-aconityl groups. In yet a further embodiment, the enzymatically cleavable linker comprises a peptide-based linker or a β-glucuronide-based linker. In a particular embodiment, a pulldown agent is used to isolate or purified the labelled newly synthesized microbial nucleic acids. In another embodiment, the pulldown reagent is an antibody immobilized onto a solid support, wherein the antibody binds with high specificity to labelling reagent, or with high specificity to the one or more types of nucleoside or nucleotide analogs. In yet another embodiment, the pulldown reagent is streptavidin or avidin immobilized onto a solid support, and wherein the labelling reagent comprises a biotin group. In another embodiment, the solid support is nano- or micro-materials, beads or a plate. In yet another embodiment, the labelling reagent or label is removed or cleaved from the isolated or purified newly synthesized microbial nucleic acids prior to (f) (f′) and (g) described above. In a further embodiment, determining the gene expression level and/or amounts and/or identity of the isolated or purified newly synthesized microbial nucleic acids in the control sample and the treated sample is determined by using a microarray comprising probes to nucleic acids from different microorganisms. In yet a further embodiment, determining the gene expression level and/or amounts and/or identity of the isolated or purified newly synthesized microbial nucleic acids in the control sample and the treated sample is by: (i) amplifying the isolated or purified newly synthesized microbial nucleic acids from the control sample using a first PCR based method using primers containing a fluorescent dye to form labelled products, wherein the primers comprise a sequence that is specific to a conserved microbial 16S rRNA gene region; (i′) amplifying the isolated or purified newly synthesized microbial nucleic acids from the treated sample using the first PCR based method using primers containing the fluorescent dye to form labelled products, wherein the primers comprise a sequence that is specific to a conserved microbial 16S rRNA gene region; (ii) applying the labelled products from the control sample to a first microarray comprising probes that comprise unique 16s rRNA variable region sequences from 20 or more microorganisms; (ii′) applying the labelled products from the treated sample to a second microarray, wherein the second microarray is a duplicate of the first microarray; and (iii) determining the effectiveness of an antimicrobial agent in modulating the growth and proliferation of microorganism(s) in a sample based upon imaging the first microarray and imaging the second microarray for fluorescent hybridization products and determining if there are any changes in regards to the intensity, location, or absence of the fluorescent hybridization products between the microarrays, wherein if there is a decrease in the intensity of the fluorescent hybridization products between the first and second microarray, or if there are changes as to the location or an absence of fluorescent hybridization products between first and second microarray indicates that the antimicrobial agent is effective in modulating the growth and proliferation of the microorganism(s). In another embodiment, the effectiveness of an antimicrobial agent in modulating the growth and proliferation of microorganism(s) in a sample is determined or confirmed by sequencing the isolated or purified newly synthesized microbial nucleic acids from the control sample and from the treated sample, wherein a decrease in the gene expression level of the newly synthesized microbial nucleic acids in the treated sample v. the control sample, or there is decrease in the amounts and/or identity of the newly synthesized microbial nucleic acids in the treated sample v. the control sample indicates that the antimicrobial agent is effective in modulating the growth and proliferation of the microorganism(s). In yet another embodiment, the isolated or purified newly synthesized microbial nucleic acids from the control and treated samples are sequenced using a transposome-based sequencing method. In a further embodiment, sequencing of the newly synthesized microbial nucleic acids from the control and treated samples are by: (a) applying the isolated or purified newly synthesized microbial nucleic acids from the control and treated samples to bead-linked transposomes, wherein the bead-linked transposomes mediate the simultaneous fragmentation of microbial nucleic acids and the addition of sequencing primers; (b) amplifying the microbial nucleic acid fragments with primers that comprise index and adapter sequences to form library of amplified products; (c) washing and pooling the library of amplified products from the control sample; (c′) washing and pooling the library of amplified products from the treated sample; (d) sequencing the libraries of amplified products from the control sample; (d′) sequencing the libraries of amplified products from the treated sample; and (e) determining any changes in the gene expression level and/or amounts and/or identity of the isolated or purified newly synthesized microbial nucleic acids from the control and treated samples based using bioinformatic analysis. In yet a further embodiment, the newly synthesized microbial nucleic acids are RNA, wherein the microbial RNA is reversed transcribed into cDNA prior to (f), (f′) and (g) described above, and wherein the effectiveness of an antimicrobial agent in modulating the growth and proliferation of microorganism(s) can be determined based upon determining changes in the gene expression levels of newly synthesized microbial nucleic acids from the control and treated samples by using a microarray and/or by sequencing.

### DESCRIPTION OF DRAWINGS

FIG. 1 presents an exemplary embodiment of a workflow for the enrichment of newly synthesized DNA from a rapid bacterial culture. The enrichment of newly synthesized DNA allows for the genetic identification of live bacteria in patient samples.

FIG. 2 presents an exemplary embodiment of a workflow for the enrichment of newly synthesized RNA from a rapid bacterial culture. The enrichment of newly synthesized RNA allows for the assessment of gene expression by live bacteria in patient samples.

### DETAILED DESCRIPTION

As used herein and in the appended claims, the singular forms “a,” “an,” and “the” include plural referents unless the context clearly dictates otherwise. Thus, for example, reference to “a microorganism” includes a plurality of such microorganisms and reference to “the nucleoside analog” includes reference to one or more nucleoside analogs and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments only and is not intended to limit the scope of the invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

The term “click chemistry,” as used herein, refers to a [3+2] cycloaddition reaction when performed in the presence of a copper (I) catalyst. The copper (I) catalyst may comprise copper(I) ions or a copper(I) chelating moiety. The copper(I) chelating moiety may be “any entity characterized by the presence of two or more polar groups that can participate in the formation of a complex (containing more than one coordinate bond) with copper(I) ions” (e.g., see Salic et al., U.S. Pat. App. No. 20070207476 (supra)). Examples of copper(I) chelating agents include, but are not limited to, neocuproine and bathocuproine disulphonate (e.g., see Salic et al., U.S. Pat. App. No. 20070207476 and Sharpless et al., US Publication No. 2003000516671). [3+2] cycloaddition reactions are also known as 1,3 dipolar cycloadditions, and may occur between 1,3-dipoles and dipolarophiles. Examples of 1,3-dipoles include azides. Examples of dipolarphiles include alkyne.

The term “dye”, as used herein, refers to a compound that emits light to produce an observable detectable signal.

The term “dual labeling”, as used herein, refers to a labeling process in which a nucleic acid is labeled with two detectable agents that produce distinguishable signals. The nucleic acid resulting from such a labeling process is said to be dually labeled.

The term “dye-labeled alkyne”, as used herein, refers to an alkyne that has been further modified to include a dye label.

The terms “dye-labeled azide” and “azide-dye molecule”, as used herein, refer to a compound or molecule with a reactive azide group that is also labeled with a dye. Examples include, but are not limited to: rhodamine-azide, Alexa Fluor® 350-azide (Molecular Probes™/Invitrogen™, Carlsbad, Calif.), Alexa Fluor® 488-azide (Molecular Probes™/Invitrogen™, Carlsbad, Calif.), Alexa Fluor® 555-azide (Molecular Probes™/Invitrogen™, Carlsbad, Calif.), Alexa Fluor® 568-azide (Molecular Probes™/Invitrogen™, Carlsbad, Calif.), Alexa Fluor® 568-azide (Molecular Probes™/Invitrogen™, Carlsbad, Calif.), Alexa Fluor® 594-azide, Alexa Fluor® 633-azide (Molecular Probes™/Invitrogen™, Carlsbad, Calif.), Alexa Fluor® 647-azide (Molecular Probes™/Invitrogen™, Carlsbad, Calif.), Cascade Blue® azide (Molecular Probes™/Invitrogen™, Carlsbad, Calif.), fluorescein-azide, coumarin-azide, BODIPY-azide, cyanine-azide, or tetramethylrhodamine (TMR)-azide.

The term “dye-labeled cycloalkyne”, as used herein, refers to a cycloalkyne that has been further modified to include a dye label. The term “cycloalkyne” refers to compounds or molecules which may be used in strained [3+2] cycloaddition reactions in order to label DNA. In this context, examples of cycloalkynes include, but are not limited to: cyclooctynes, difluorocyclooctynes, heterocycloalkynes, dichlorocyclooctynes, dibromocyclooctynes, or diiodocyclooctynes.

The term “effective amount”, as used herein, refers to the amount of a substance, compound, molecule, agent or composition that elicits the relevant response in a cell, a tissue, or a microorganism. For example, in the case of microorganisms contacted with a nucleoside analog, an effective amount is an amount of nucleoside that is incorporated into the DNA of the microorganisms.

The term “fluorophore” or “fluorogenic”, as used herein, refers to a composition that demonstrates a change in fluorescence upon binding to a biological compound or analyte interest. Preferred fluorophores of the present disclosure include fluorescent dyes having a high quantum yield in aqueous media. Exemplary fluorophores include xanthene, indole, borapolyazaindacene, furan, and benzofuran, cyanine among others. The fluorophores of the present invention may be substituted to alter the solubility, spectral properties or physical properties of the fluorophore.

The term “label”, as used herein, refers to a chemical moiety or protein that retains its native properties (e.g., spectral properties, conformation and activity) when part of a labeling reagent of the disclosure and used in the methods of the disclosure. Illustrative “label” molecules can be directly detectable (fluorophore), indirectly detectable (hapten or enzyme), or could be used for detection and purification of nucleoside incorporated nucleic acids (e.g., biotin-streptavidin pull-down assay). Such “label” molecules include, but are not limited to, click chemistry designed biotin labels, iminobiotin or desthiobiotin containing labels, such as

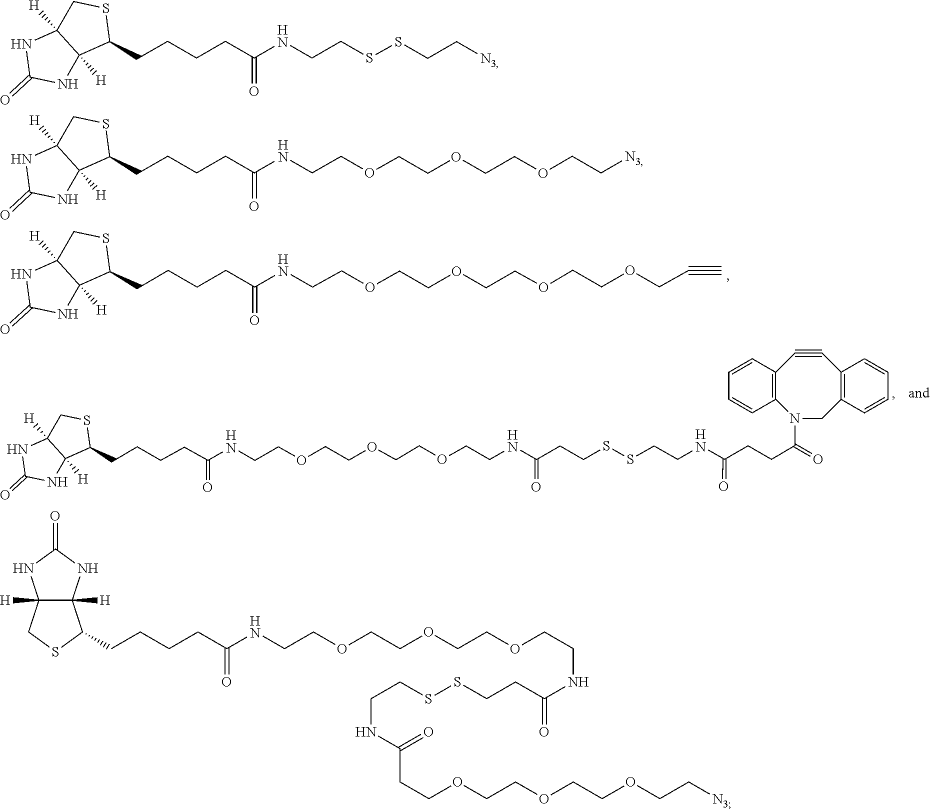

radio reporter molecules that can be measured with radiation-counting devices; pigments, dyes or other chromogens that can be visually observed or measured with a spectrophotometer; spin labels that can be measured with a spin label analyzer; fluorescent moieties, where the output signal is generated by the excitation of a suitable molecular adduct and that can be visualized by excitation with light that is absorbed by the dye or can be measured with standard fluorometers or imaging systems, for example. The “label” molecule can be a luminescent substance such as a phosphor or fluorogen; a bioluminescent substance; a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The “label” may also take the form of a chemical or biochemical, or an inert particle, including but not limited to colloidal gold, microspheres, quantum dots, or inorganic crystals such as nanocrystals or phosphors (e.g., see Beverloo et al., Anal. Biochem. 203, 326-34 (1992)). The “label” molecule can also be a “tag” or hapten that is used to “tag” the nucleoside analog. The “tag” can then be bound by another reagent that selectively binds to the “tag.” For instance, one can use biotin, iminobiotin or desthiobiotin as a “tag” and then use avidin or streptavidin conjugated to a substrate (e.g., beads), a label, or enzyme (e.g., horse radish peroxidase), to bind to the biotin-based “tag”. In regards to the latter, a chromogenic substrate (e.g., tetramethylbenzidine) or a fluorogenic substrate such as Amplex Red or Amplex Gold (Molecular Probes, Inc.) can then be used. In a similar fashion, the tag can be a hapten or antigen (e.g., digoxigenin), and an enzymatically, fluorescently, or radioactively labeled antibody can be used to bind to the tag. Numerous reporter molecules are known by those of skill in the art and include, but are not limited to, particles, fluorescent dyes, haptens, enzymes and their chromogenic, fluorogenic, and chemiluminescent substrates, and other reporter molecules that are described in the MOLECULAR PROBES HANDBOOK OF FLUORESCENT PROBES AND RESEARCH CHEMICALS by Richard P. Haugland, 10th Ed., (2005).

The term “microorganism” or “microbe” are used herein interchangeably and refer to a microscopic organism, which may exist in its single-celled form or in a colony of cells. For purposes of this disclosure, “microorganism” as used herein includes bacteria, fungi, viruses, algae, archaea, and protozoa.

The term “microbial proliferation” as used herein refers to an expansion and/or growth of microorganism(s).

The term “nucleoside analog” and “nucleotide analog” are used interchangeably and refer herein to a molecule or compound that is structurally similar to a natural nucleoside or nucleotide that is incorporated into newly synthesized microbial nucleic acid. In the case of nucleosides, once inside the cells, they are phosphorylated into nucleotides and then incorporated into nascent nucleic acid polymers. Nucleotides are difficult to get across the cell membrane due to their charges and are more labile than nucleosides, thus their use typically requires and additional step and reagents for transfection to transport the nucleotides across the lipid bilayer. The present nucleoside analogs are incorporated into nucleic acid (DNA or RNA) in a similar manner as a natural nucleotide wherein the correct polymerase enzyme recognizes the analogs as natural nucleotides and there is no disruption in synthesis. These analogs comprise a number of different moieties which are ultimately used for detection, such as halogenated analogs (bromo, chloro, iodo, etc.) and those that comprise a bioorthogonal moiety such as azido, alkyne or phosphine.

The term “pulldown reagent”, as used herein, refers to a reagent that is used to purify or isolate a nascent nucleic acid polymer which comprises one or more labelled nucleotide analogs disclosed herein. The “pulldown reagent” is typically bound to a solid support, such as beads, and selectively binds with the label disclosed herein. Typically, the label functions as a “tag” as described above. In an exemplary embodiment, the pulldown reagent is a streptavidin conjugated to a solid support, such as beads, superparamagnetic micro- or nano-particles, a plate, etc. In another embodiment, the pulldown reagent is an antibody or other type of affinity ligand that is specific for the label or “tag” that is immobilized on a solid support, such as beads, superparamagnetic micro- or nano-particles, a plate, etc.

The term “Staudinger ligation”, as used herein, refers to a chemical reaction developed by Saxon and Bertozzi (E. Saxon and C. Bertozzi, Science, 2000, 287: 2007-2010) that is a modification of the classical Staudinger reaction. The classical Staudinger reaction is a chemical reaction in which the combination of an azide with a phosphine or phosphite produces an aza-ylide intermediate, which upon hydrolysis yields a phosphine oxide and an amine. A Staudinger reaction is a mild method of reducing an azide to an amine; and triphenylphosphine is commonly used as the reducing agent. In a Staudinger ligation, an electrophilic trap (usually a methyl ester) is appropriately placed on a triarylphosphine aryl group (usually ortho to the phosphorus atom) and reacted with the azide, to yield an aza-ylide intermediate, which rearranges in aqueous media to produce a compound with amide group and a phosphine oxide function. The Staudinger ligation is so named because it ligates (attaches/covalently links) the two starting molecules together, whereas in the classical Staudinger reaction, the two products are not covalently linked after hydrolysis.

The terms “subject”, “patient” and “individual” are used interchangeably herein, and refer to an animal, particularly a human, from whom a sample may be obtained. This includes human and non-human animals. The term “non-human animals” and “non-human mammals” are used interchangeably herein includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. A subject can be male or female. A subject can be a fully developed subject (e.g., an adult) or a subject undergoing the developmental process (e.g., a child, infant or fetus).

Living, proliferating microorganisms (e.g., bacteria algae, archaea, protozoa, and fungi) continuously synthesize new DNA. In direct contrast, microorganisms that are no longer viable will no longer synthesize DNA. While it is possible that live, non-proliferating microorganisms synthesize new DNA to repair and maintain their genomes, the rate of new DNA synthesis will be far lower than living, proliferating microorganisms. The disclosure provides for methodologies and technologies that utilize the foregoing differences in DNA synthesis in order to expeditiously identify the living, proliferating microorganisms in a sample, such as a blood sample from a patient, or an environmental sample; or a sample from a suspected contaminated foodstuff. In particular the methodologies and technologies presented herein allow for identification of living, proliferating microorganisms in a sample, irrespective of whether the sample further comprises or is contaminated with non-viable or non-proliferating microorganisms. More specifically, the methodologies and technologies presented herein provide for the selective enrichment and sequencing of newly synthesized microbial DNA obtained from one or more microorganisms in a sample, allowing for identification of the living, proliferating microorganisms contained in the sample.

The disclosure also provides methods and composition that can be used to selectively enrich for DNA and RNA from a specific organism or similar group of organisms in a mixed population. For example, for dehosting applications, one desires to enrich for the DNA or RNA of an infectious organism from a background of DNA or RNA from the infected host. The methods allow for rapid enrichment of DNA and/or RNA of targeted organisms (e.g., bacteria) from a mixed population in order to identify the targeted organism. The enrichment requires conditions so that only the targeted organisms are able to synthesize DNA and/or RNA. For example, media conditions, temperature and/or specific inhibitors can be used to selectively inhibit a targeted population or sub-population in a sample.

As an example, blood from a subject having or suspected of having sepsis can be obtained and cultured under conditions whereby the mammalian cells in the blood sample are inhibited from DNA and/or RNA synthesis while bacterial cells in the sample can continue to synthesize DNA and/or RNA. In this manner the bacterial DNA and/or RNA is selectively labeled. In one embodiment, a blood sample can be isolated and plated or cultured in LB broth or other bacterial mediums such that the mammalian cells will not continue to undergo DNA and/or RNA synthesis (or have substantially reduced DNA and/or RNA synthesis), while at the same time the microbial population will continue to undergo DNA and RNA synthesis leading to selective incorporation of, for example, EdU. In another embodiment, the temperature of a blood culture can be lowered whereby mammalian cell replication and synthesis will be inhibited while only microbial replication and synthesis will be maintained or renewed upon returning to a higher temperature. The temperature can be lowered over a period of time from several minutes to several hours. In another embodiment, a small molecule inhibitor of DNA and/or RNA synthesis can be used that selectively targets mammalian DNA and/or RNA machinery. For example, one inhibitor is derived from the Amanita mushroom, called alpha-amanitin, and is responsible for about a hundred deaths annually among undiscriminating mushroom hunters. RNAP inhibitors can be specific for a single class of organisms. Alpha-amanitin, for example, affects higher eukaryotes, but has no effect on bacteria. Conversely, some drugs specifically affect bacterial RNAP. The best known of these is rifampin, which is produced by a fungi and is currently in use as an anti-tuberculosis drug as the rifampin derivative Rifampicin (Rif). Rif is specific for bacterial RNAPs. This specificity of inhibitors occurs for two reasons. First, the inhibitors are often made by one organism to kill another and the producing organism must evolve an inhibitor that is not suicidal. Second, the inhibitors usually bind to the less-conserved parts of the enzyme, where sequence variation can prevent them from working on all RNAPs.

The disclosure also provides embodiments directed to dehosting a sample prior to the identification of nascent microbial nucleic acid synthesis using the methods of the disclosure. Such dehosting techniques and compositions relate to, for example, the selective cleavage of non-microbial nucleic acids in a sample containing both microbial and non-microbial nucleic acids, so that the sample becomes greatly enriched with microbial nucleic acids. Examples of dehosting methods include those described in Feehery et al., PLoS ONE 8:e76096 (2013); Sachse et al., Journal of Clinical Microbiology 47:1050-1057 (2009); Barnes et al., PLoS ONE 9(10):e109061 (2014); Leichty et al., Genetics 198(2):473-81 (2014)); Hasan et al., J Clin Microbiol 54(4):919-27 (2016); and Liu et al., PLoS ONE 11(1):e0146064 (2016); the disclosures of which are incorporated herein in-full. Additionally, commercial kits for carrying out dehosting are also available, including the NEBNext Microbiome DNA Enrichment™ Kit, the Molzym MolYsis Basic™ kit, and MICROBEEnrich™ Kit.

In some embodiments, the dehosting methods and compositions disclosed herein takes advantage of properties associated with nonmicrobial nucleic acids, including methylation at CpG residues, and associations with DNA-binding proteins, such as histones. For example, in a particular embodiment the dehosting methods and compositions can utilizes a nucleic acid binding protein that selectively binds with nonmicrobial nucleic acids (e.g., histones, restriction enzymes). In a further embodiment, the dehosting methods and compositions can comprise a recombinant protein that selectively binds with nonmicrobial nucleic acids, and which also selectively degrades nonmicrobial nucleic acids, i.e., the recombinant protein comprises both a nonmicrobial nucleic acid binding domain and a nuclease domain. In a particular embodiment, the nucleic acid binding protein is a histone. Histones are found in the nuclei of eukaryotic cells, and in certain Archaea, namely Thermoproteales and Euryarchaea, but not in bacteria or viruses. In a further embodiment, histone bound nonmicrobial nucleic acids can then be removed from the sample by use of a substrate which comprises an affinity agent that selectively binds to a histone protein, i.e., a histone-binding domain. Examples of affinity agents that can bind to a histone protein include, but are not limited to, chromodomain, Tudor, Malignant Brain Tumor (MBT), plant homeodomain (PHD), bromodomain, SANT, YEATS, Proline-Tryptophan-Tryptophan-Proline (PWWP), Bromo Adjacent Homology (BAH), Ankryin repeat, WD40 repeat, ATRX-DNMT3A-DNMT3L (ADD), or zn-CW. In another embodiment, the histone-binding domain can include a domain which specifically binds to a histone from a protein such as HAT1, CBP/P300, PCAF/GCN5, TIP60, HBO1 (ScESAl, SpMST1), ScSAS3, ScSAS2 (SpMST2), ScRTT109, SirT2 (ScSir2), SUV39H1, SUV39H2, G9a, ESET/SETDB1, EuHMTase/GLP, CLL8, SpClr4, MLL1, MLL2, MLL3, MLL4, MLL5, SET1A, SET1B, ASH1, Sc/Sp SET1, SET2 (Sc/Sp SET2), NSD1, SYMD2, DOT1, Sc/Sp DOT1, Pr-SET 7/8, SUV4 20H1, SUV420H2, SpSet 9, EZH2, RIZ1, LSD1/BHC110, JHDM1a, JHDM1b, JHDM2a, JHDM2b, JMJD2A/JHDM3A, JMJD2B, JMJD2C/GASC1, JMJD2D, CARM1, PRMT4, PRMT5, Haspin, MSK1, MSK2, CKII, Mstl, Bmi/Ring1A, RNF20/RNF40, or ScFPR4, or a histone-binding fragment thereof.

In additional embodiment, the disclosure also provides for a nucleic acid binding protein or nucleic acid binding domain that selectively binds to DNA that comprises a methylated CpG. CG dinucleotide motifs (“CpG sites” or “CG sites”) are found in regions of DNA where a cytosine nucleotide is followed by a guanine nucleotide in the linear sequence of bases along its 5′ to 3′ direction. CpG islands (or CG islands) are regions with a high frequency of CpG sites. CpG is shorthand for 5′-C-phosphate-G-3′, that is, cytosine and guanine separated by one phosphate. Cytosines in CpG dinucleotides can be methylated to form 5-methylcytosine. Cytosine methylation occurs throughout the human genome at many CpG sites. Cytosine methylation at CG sites also occurs throughout the genomes of other eukaryotes. In mammals, for example, 70% to 80% of CpG cytosines may be methylated. In microbes of interest, such as bacteria and viruses, this CpG methylation does not occur or is significantly lower than the CpG methylation in the human genome. Thus, dehosting can be achieved by selectively cleaving CpG methylated DNA.

In some embodiments, the disclosure provides for a dehosting method which comprises a nucleic acid binding protein or binding domain which binds to CpG islands or CpG sites. In another embodiment, the binding domain comprises a protein or fragment thereof that binds to methylated CpG islands. In yet another embodiment, the nucleic acid binding protein binding domain comprises a methyl-CpG-binding domain (MBD). An example of an MBD is a polypeptide of about 70 residues that folds into an alpha/beta sandwich structure comprising a layer of twisted beta sheet, backed by another layer formed by the alpha1 helix and a hairpin loop at the C terminus. These layers are both amphipathic, with the alpha1 helix and the beta sheet lying parallel and the hydrophobic faces tightly packed against each other. The beta sheet is composed of two long inner strands (beta2 and beta3) sandwiched by two shorter outer strands (beta1 and beta4). In a further embodiment, the nucleic acid binding protein or binding domain comprises a protein selected from the group consisting of MECP2, MBD1, MBD2, and MBD4, or a fragment thereof. In yet a further embodiment, the nucleic acid binding protein or binding domain comprises MBD2. In a certain embodiment, the nucleic acid binding protein or binding domain comprises a fragment of MBD2. In another embodiment, the nucleic acid binding protein or binding domain comprises MBD5, MBD6, SETDB1, SETDB2, TIP5/BAZ2A, or BAZ2B, or a fragment thereof. In yet another embodiment, the nucleic acid binding protein or binding domain comprises a CpG methylation or demethylation protein, or a fragment thereof. In a further embodiment, CpG bound nonmicrobial nucleic acids can then be removed from the sample by use of a substrate which comprises an affinity agent that selectively binds to a nucleic acid binding protein or binding domain which binds to CpG islands or CpG sites. Examples of affinity agents include antibodies or antibody fragments that selectively bind to a nucleic acid binding protein or binding domain which binds to CpG islands or CpG sites. Affinity agents comprising antibodies or antibody fragments can be bound to a substrate or alternatively may itself be bound by a second antibody which is bound to a substrate, thereby providing a means to separate and remove the nonmicrobial nucleic acids from a sample.

In another embodiment the disclosure provides for dehosting method that uses a nuclease, or a recombinant protein which comprises a nuclease domain, whereby the nuclease cleaves nonmicrobial nucleic acids into fragments. In the latter case, the recombinant protein may also comprise a nucleic acid protein binding domain having activity for nucleic acid binding proteins (e.g., histones, methyl-CpG-binding proteins). The nuclease or nuclease can include, but are not limited to, a non-specific nuclease, an endonuclease, non-specific endonuclease, non-specific exonuclease, a homing endonuclease, and restriction endonuclease. In another embodiment, the nuclease domain is derived from any nuclease where the nuclease or nuclease domain does not itself have its own unique target. In yet another embodiment, the nuclease domain has activity when fused to other proteins. Examples of non-specific nucleases include FokI and I-TevI. In some embodiments, the nuclease domain is FokI or a fragment thereof. In a further embodiment, the nuclease domain is I-TevI or a fragment thereof. In yet a further embodiment, the FokI or I-TevI or fragment thereof is unmutated and/or wild-type. Further examples of nucleases include but are not limited to, Deoxyribonuclease I (DNase I), RecBCD enonuclease, T7 endonuclease, T4 endonuclease IV, Bal 31 endonuclease, endonucleasel (endo I), Micrococcal nuclease, Endonuclease II (endo VI, exo III), Neurospora endonuclease, S1-nuclease, P1-nuclease, Mung bean nuclease I, Ustilago nuclease (Dnase I), AP endonuclease, and Endo R.

The microorganisms of interest could be identified in a variety of samples, including but not limited to, samples from patients (e.g., blood, urine, and spinal fluid), foodstuff samples (e.g., flour, beef, and lettuce), or environmental samples (e.g., ground water, and hospital building swabs). A main advantage of the methods, compositions and kits disclosed herein is that viable, and/or proliferating microorganism(s) in a sample can be identified without needing to extensively culture the microorganism prior to identification. Thus, microorganisms, like Treponema pallidum (Syphilis) and environmental bacteria, which cannot be cultured in vitro on routine culture media or in tissue culture, can be readily identified using the methods, compositions and kits of the disclosure.

Further, disclosed herein are methods for labelling, purifying, and sequencing newly synthesized nucleic acids in order to identify, and analyze viable microorganisms in a patient, food, environmental or other sample. The methods of the disclosure can be further used to screening test compounds (e.g., antibiotics) for their effect on the viable microorganisms identified in the sample. The methods disclosed herein utilize nucleoside analogs that are “fed” to the microorganisms and incorporated into newly synthesized or nascent nucleic acids. In regards to microorganisms, any type of microorganism can be detected by the methods disclosed herein, including bacteria, fungi, viruses, algae, archaea, and protozoa.

Bacteria are prokaryotes that lack a nucleus and contain no organelles. Within the bacteria family there are two classes, Gram positive bacteria which have thicker cell wall and Gram negatives which have a thinner layer sandwiched between an inner and outer membrane. Bacteria are extremely diverse and in terms of number are by far the most successful organism on Earth. Bacteria are the only microorganisms which can live harmlessly within the human body, often aiding bodily functions such as digestion. Outside of viruses, bacteria cause the most problems in terms of disease in humans, such as sepsis. Examples of bacteria that can be identified and analyzed using the methods, compositions and kits disclosed herein, include, but are not limited to, Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

Fungi are eukaryotes which means they have a defined nucleus and organelles. The cells are larger than prokaryotes such as bacteria. Fungal colonies can be visible to the human eye once they have achieved a certain level of growth, for example mould on bread. Fungi can be split into three main groups, (1) moulds which display thread-like (filamentous) growth and multicellular structures, (2) yeasts which are typically non-filamentous and can be single celled, and (3) mushrooms which possess a fruiting body for production of spores. Fungi can be problematic for the immunocompromised and can be significant pathogens for plants. Examples of fungi that can be identified and analyzed using the methods, compositions and kits disclosed herein, include, but are not limited to, Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp., Blastomyces spp., Cadophora spp, Candida albicans, Cercospora apii, Chrysosporium spp., Cladosporium spp., Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp., Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp., Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.

Viruses represent a large group of submicroscopic infective agents that are usually regarded as nonliving extremely complex molecules, that typically contain a protein coat surrounding an RNA or DNA core of genetic material but no semipermeable membrane, that are capable of growth and multiplication only in living cells, and that cause various important diseases in humans, animals, and plants. Examples of viruses that can be identified and analyzed using the methods, compositions and kits disclosed herein, include, but are not limited to, Simplexvirus, Varicellovirus, Cytomegalovirus, Roseolovirus, Lympho-cryptovirus, Rhadinovirus, Mastadenovirus, α-Papillomavirus, β-Papillomavirus, X-Papillomavirus, γ-Papillomavirus, Mupapillomavirus, Nupapillomavirus, Alphapolyomavirus, Betapolyomavirus, γ-Polyomavirus, Deltapolyomavirus, Molluscipoxvirus, Orthopoxvirus, Parapoxvirus, α-Torquevirus, β-Torquevirus, γ-Torquevirus, Cyclovirus, Gemycircular, Gemykibivirus, Gemyvongvirus, Erythrovirus, Dependovirus, Bocavirus, Orthohepadnavirus, Gammaretrovirus, Deltaretrovirus, Lentivirus, Simiispumavirus, Coltivirus, Rotavirus, Seadornavirus, α-Coronavirus, β-Coronavirus, Torovirus, Mamastrovirus, Norovirus, Sapovirus, Flavivirus, Hepacivirus, Pegivirus, Orthohepevirus, Cardiovirus, Cosavirus, Enterovirus, Hepatovirus, Kobuvirus, Parechovirus, Rosavirus, Salivirus, Alphavirus, Rubivirus, Ebolavirus, Marburgvirus, Henipavirus, Morbilivirus, Respirovirus, Rubulavirus, Metapneumovirus, Orthopneumovirus, Ledantevirus, Lyssavirus, Vesiculovirus, Mammarenavirus, Orthohantavirus, Orthonairovirus, Orthobunyavirus, Phlebovirus, α-Influenzavirus, β-Influenzavirus, γ-Influenzavirus, Quaranjavirus, Thogotovirus, and Deltavirus.

Algae are a more difficult to define group of organisms, containing both prokaryotes and eukaryotes by some definitions. Unlike other microorganisms, algae are typically photosynthesizers and are typically found in marine environments. Harmful algal blooms (HABs) are an algal bloom that causes negative impacts to other organisms via production of natural toxins, mechanical damage to other organisms, or by other means. HABs are often associated with large-scale marine mortality events and have been associated with various types of shellfish poisonings. HABs involve toxic or otherwise harmful phytoplankton such as dinoflagellates of the genus Alexandrium and Karenia, or diatoms of the genus Pseudo-nitzschia. Such blooms often take on a red or brown hue and are known colloquially as red tides. The methods, compositions and kits of the disclosure allow for identification of such algae from samples, e.g., environmental samples.

Archaea are prokaryotes that have a similar morphology to bacteria. Archaea differ from eukarya and bacteria in terms of genetic, biochemical, and structural features. For example, archaea possess unique flagellins and ether-linked lipids and lack murein in their cell walls. Archaea share some characteristics with known pathogens that may reflect the potential to cause disease. Such characteristics include ample access to a host (i.e., opportunity) and capabilities for long-term colonization and coexistence with endogenous flora in a host. The detection of anaerobic archaea in the human colonic, vaginal, and oral microbial flora demonstrates their ability to colonize the human host. The methods, compositions and kits of the disclosure allow for identification of such archaea from samples, e.g., environmental samples, samples obtained from a subject, etc.

Protozoa refers to single-celled eukaryotes, either free-living or parasitic, which feed on organic matter such as other microorganisms or organic tissues and debris. Protozoa, as traditionally defined, range in size from as little as 1 micrometer to several millimeters, or more. All protozoans are heterotrophic, deriving nutrients from other organisms, either by ingesting them whole or consuming their organic remains and waste-products. Some protozoans take in food by phagocytosis, engulfing organic particles with pseudopodia (as amoebae do), or taking in food through a specialized mouth-like aperture called a cytostome. Others take in food by osmotrophy, absorbing dissolved nutrients through their cell membranes. A number of protozoan pathogens are human parasites, causing diseases such as malaria (by Plasmodium), amoebiasis, giardiasis, toxoplasmosis, cryptosporidiosis, trichomoniasis, Chagas disease, leishmaniasis, African trypanosomiasis (sleeping sickness), amoebic dysentery, acanthamoeba keratitis, and primary amoebic meningoencephalitis (naegleriasis). The methods, compositions and kits of the disclosure allow for identification of such protozoa from samples e.g., environmental samples, samples obtained from a subject, etc.

The methods of the disclosure provide for identification and analysis of the foregoing microorganisms from a sample, in particular, microorganisms that are viable and/or proliferating. As indicated above, the sample can originate from a variety of sources, including from subjects, from the environment, from foodstuffs, etc. Any number of types of samples from subjects can be used with the compositions, methods, and kits of the disclosure, including, but not limited to, blood, urine, saliva, middle ear aspirate, bile, vaginal secretions, pus, pleural effusions, synovial fluid, and abdominal cavity abscesses. As such, the methods, compositions and kits of the disclosure are not particular limited by the type and location of the sample obtained from a subject. Moreover, the methods, kits and compositions disclosed herein provide an improvement over standard methodologies in identifying microorganisms that are causing sepsis in a patient, or causing urinary infection in a patient, in that the methods, kits and compositions disclosed herein can accurately identify the offending microorganisms in much more rapid manner than the standard methodologies. As such, the appropriate antimicrobial(s) for the identified microorganism(s) can be administered much sooner, thereby fighting and clearing a microbial infection in a more expeditious manner and possible preventing or lessening side effects associated with the microbial infection, such as septic shock, chills, fever, body aches, changes in mental ability, fatigue, malaise, breathing problems, abnormal heart infections, inflammation, nausea and vomiting, anxiety, etc. Moreover, the methods, kits and compositions disclosed herein can also determine if an antimicrobial agent is effective in inhibiting or killing a microorganism. Thus, if the microorganism is resistant to a particular antimicrobial, the methods, kits and compositions disclosed herein can make such a determination in an expeditious manner, so that another antimicrobial can tried.

The methods, compositions and kits of the disclosure can utilize both nucleoside and nucleotide analogs for identifying nascent microbial nucleic acid synthesis. As described more fully below, the methods, compositions and kits of the disclosure can utilize multiple types of nucleoside and nucleotide analogs, and the use of which can be advantageous for establishing base line nucleic acid synthesis, and determining changes in the rate of nucleic acid synthesis, such as the addition of antimicrobial agent. Nucleosides are typically used in experiments wherein the analogs are added to cell culture or administered to animals because the nucleoside analogs are easily taken up by live cells, wherein they are phosphorylated into a nucleotide and then incorporated into a growing nucleic acid polymer. In contrast, nucleotides are more labile and more susceptible to enzyme cleavage, either before or after incorporation into cells, and are generally less stable than nucleosides. In addition, due to the additional charges from the phosphate groups, nucleotides are not easily transported into live cells and generally require a transfection step to get a sufficient concentration of nucleotides across the cellular membrane. This is not ideal for either in vivo or ex vivo/in vivo experiments where cell perturbation should be kept to a minimum to accurately interpret results. For these reasons, the following disclosure generally refers to nucleosides as the analog that is added to cells or animals, however this in no way is intended to be limiting, wherein nucleotides are equally as important.

The nucleoside and nucleotide analogs can be an analog for any of the four DNA bases (adenine (A), cytosine (C), guanine (G) or thymine (T)) or any of the four RNA bases (adenine (A), cytosine (C), guanine (G) or uracil (U)) and include their triphosphate and phosphoramidite forms. Nucleoside and nucleotide analogs are incorporated into newly synthesized nucleic acid by polymerases present in the microorganisms. Nucleoside and nucleotide analogs are different from the naturally occurring nucleosides in that the phosphate backbone, the pentose sugar, and/or the ribose or deoxyribose have been altered, typically by synthetic chemistry techniques, e.g., the nucleotide or nucleoside may be altered to comprise a detectable label (e.g., a dye, a fluorophore), a bioorthogonal functional moiety (e.g., a moiety that is involved in particular chemical reactions, like click chemistry), a biomolecule (e.g., an enzyme, antibody, biotin), etc., any one of which, can be used in the methods, compositions and kits of the disclosure to identify nascently made microbial nucleic acid polymers. In one embodiment the nucleoside analog is a halogenated analog, including but not limited to a bromo, chloro, and iodo moiety. Examples of halogenated analogs include, but are not limited to, 2′ (S)-2′-deoxy-2′-fluoro-5-ethynyluridine, (2'S)-2′-fluoro-5-ethynyluridine, 5-bromo-2′deoxyuridine, 5-bromouridine, 5-iodo-2′deoxyuridine, and 5-iodouridine. In regards to the halogenated analogs, antibodies have been specifically developed to bind with high affinities to these analogs, like bromo-2′deoxyuridine and iododeoxyuridine (see Dako, Carpinteria, Calif.; BD Bioscience, San Diego, Calif.; EMD Biosciences, Madison, Wis.). In another embodiment the nucleoside or nucleotide analog comprises a bioorthogonal functional moiety, including but not limited to an azido, alkynyl or phosphinyl moiety. Examples of nucleoside or nucleotide analogs comprising a bioorthogonal functional moiety include, but are not limited to, 2-ethynyl-adenosine, N6-propargyl-adenosine, 2′-(O-propargyl)-adenosine, 3′-(O-propargyl)-adenosine, 5-ethynyl-cytidine, 5-ethynyl-2′-deoxycytidine, 2′-(O-propargyl)-cytidine, 3′-(O-propargyl)-cytidine, 2′-(O-propargyl)-guanosine, 3′-(O-propargyl)-guanosine, 5-ethynyl-uridine, 5-ethynyl-2′-deoxyuridine, 2′-(O-propargyl)-uridine, 3′-(O-propargyl)-uridine, (2'S)-2′-deoxy-2′-fluoro-5-ethynyluridine, (2'S)-2′-fluoro-5-ethynyluridine, 8-azido-adenosine, N⁶-(6-azido)hexyl-2′deoxy-adenosine, 2′-azido-2′-deoxyadenosine, 5-azidomethyl-uridine, 5-(15-azido-4,7,10,13-tetraoxa-pentadecanoyl-aminoallyl)-2′-deoxyuridine, 5-(3-azidopropyl)-uridine, 5-azido-PEG₄-uridine, 5-azido-PEG₄-cytidine, and 5-azido-PEG₄-2′-deoxycytidine.

In a particular embodiment, the nucleoside analog comprises bioorthogonal functional moiety that can undergo either click chemistry, a strained [3+2] cycloaddition reaction, or Staudinger ligation with a functional group of the labelling reagent. In some embodiments, the reactive bioorthogonal moiety is carried by the base of the nucleoside. The base carrying the reactive bioorthogonal moiety can be a purine (e.g., adenine or guanine) or a pyrimidine (e.g., cytosine, uracil or thymine). In certain embodiments, the base is uracil; in some such embodiments, uracil carries the reactive bioorthogonal moiety on the 5-position. In certain embodiments, the base is adenine; in some such embodiments, adenine carries the reactive bioorthogonal moiety. In certain embodiments, the bioorthogonal moiety is indirectly attached to the base, while in other embodiments the bioorthogonal moiety is directly covalently attached to the base. In certain embodiments, the reactive bioorthogonal moiety is carried by the sugar (ribose and deoxyribose) of the nucleoside. In certain embodiments, the bioorthogonal moiety is indirectly attached to the sugar, while in other embodiments the bioorthogonal moiety is directly and covalently attached to the sugar. In certain embodiments, the reactive bioorthogonal moiety attached to the phosphate moiety of the nucleoside. The sugar carrying the reactive bioorthogonal moiety can be covalently attached to a purine (e.g., adenine or guanine) or a pyrimidine (e.g., cytosine, uracil or thymine). In certain embodiments, the base is uracil, while in other embodiments the base is adenine.

The reactive bioorthogonal moiety can be a 1,3-dipole such as a nitrile oxide, an azide, a diazomethane, a nitrone or a nitrile imine. In certain embodiments, the 1,3-dipole is an azide. Alternatively, the reactive bioorthogonal functional moiety can be a dipolarophile such as an alkene (e.g., vinyl, propylenyl, and the like) or an alkyne (e.g., ethynyl, propynyl, and the like). In certain embodiments, the dipolarophile is an alkyne, such as, for example, an ethynyl group.

These bioorthogonal functional moieties described above are non-native, nonperturbing bioorthogonal chemical moieties that possess unique chemical functionality that can be modified through highly selective reactions. In particular these incorporated nucleosides are labeled using labeling reagents which comprise a chemical handle that will selectively form a covalent bond with the nucleoside in the presence of the cellular milieu.

Dissecting complex cellular processes, including microbial proliferation, requires the ability to track biomolecules as they function within their native habitat. In recent years, bioorthogonal functional moieties have been used as an additional method for tagging biomolecules. The use of bioorthogonal functional moieties has been described for the detection of metabolites and post-translational modifications using the azide moiety as a bioorthogonal functional moiety. Once introduced into target biomolecules, either metabolically or through chemical modification, the azide can be tagged with probes using one of three highly selective reactions: the Staudinger ligation, the Cu(I)-catalyzed azide-alkyne cycloaddition, or the strain-promoted [3+2] cycloaddition (e.g., see Agard et al., J Am Chem Soc. 2004 Nov. 24; 1 26(46):1 5046-7).

The bioorthogonal functional moieties can be used to label nucleic acid through the incorporation of nucleoside or nucleotide analogs. Thus, one can label nucleic acids using bioorthogonal labeling such as the Staudinger ligation, Cu(I)-catalyzed [3+2] cycloaddition of azides and alkynes (“click chemistry”) or “copper-less” click chemistry independently described by Barry Sharpless and Carolyn Bertozzi (e.g., see Sharpless et al., Angew Chem Int Ed Engl. 2002 Mar. 15; 41 (6):1 053-7; Meldal et al., J. Org. Chem. 2002, 67, 3057; Agard et al., J Am Chem Soc. 2004 Nov. 24; 1 26(46):1 5046-7; U.S. Pat. No. 7,122,703; US Publication No. 2003000516671). Click chemistry and the Staudinger ligation have been adapted to measure cellular proliferation through the direct detection of nucleotide incorporation. See Salic, et al., Methods and Compositions for Labeling Nucleic Acids, U.S. Publication No. 20070207476 and 20070099222 (filed Oct. 27, 2006).

Click chemistry techniques to label nucleic acids involve treating a cell with a first nucleoside or nucleotide analog containing a reactive unsaturated group, such that the first nucleoside analog is incorporated into newly synthesized microbial nucleic acids. Then, the cell is contacted with a labeling reagent comprising a second reactive unsaturated group attached to a label, such that a [3+2] cycloaddition occurs between the first and second reactive unsaturated groups.

The following descriptions of [3+2] cycloaddition reactions to label microbial nucleic acids are provided as examples only and are not intended to limit the scope of the present invention.

As one example of labeling microbial DNA using click chemistry, samples are treated with an effective amount of an alkyne-modified nucleoside analog, for example, 5-ethynyl-2′-deoxyuridine (EdU), for a defined period of time such that the EdU is incorporated into newly synthesized DNA. After being labeled with EdU, the labeled microbial DNA is reacted, in the presence of a copper(I) catalyst, with an azide-disulfide-biotin linker. A covalent bond is formed between the azide and the incorporated nucleoside analog, via a [3+2] cycloaddition reaction, and the resulting complex may then be captured using a streptavidin-conjugated substrate (e.g., beads). After washing the substrate, the microbial DNA is freed from the substrate by the addition of reducing agents, such as dithiothreitol (DTT). The sequence of the microbial DNA can then be determined using standard methods (e.g., Illumina Nextera DNA Flex with PCR library amplification).

In a second example of labeling microbial DNA using click chemistry, samples are treated with an effective amount of an azide-modified nucleoside analog, for example, 5-azido-2′-deoxyuracil (AzdU), for a defined period of time such that AzdU is incorporated into the newly synthesized microbial DNA. After labeling with AzdU, the labeled microbial DNA is reacted, in the presence of a copper(I) catalyst, with a dye-labeled alkyne. As a result of a [3+2] cycloaddition reaction between the azide and alkyne moieties, a covalent bond is formed. The dye label may then be measured using standard methods, including, but not limited to, flow cytometry, fluorescence microscopy, imaging, multi-well plate assays, or high content screening.

In an example of labeling RNA using click chemistry, samples are incubated in the presence of an effective amount of an alkyne-modified nucleoside analog, for example, 5-ethynyl-uridine (EU), for a defined period of time such that the EU is incorporated into newly synthesized microbial RNA. After being labeled with EU, the microbes are lyzed and reacted, in the presence of a copper(I) catalyst, with an azide-disulfide-biotin linker. A covalent bond is formed between the azide and the incorporated nucleoside analog, via a [3+2] cycloaddition reaction, and the resulting complex may then be captured using a streptavidin-conjugated substrate (e.g., beads). After washing the substrate, the RNA is freed from the substrate by the addition of reducing agents, such as dithiothreitol (DTT). The RNA is reverse transcribed into cDNA. From the cDNA, sequencing libraries can be prepared.

One alternative to click chemistry, which takes advantage of strained [3+2] cycloaddition reactions without using a copper(I) catalyst, has been described by Bertozzi et al. is the “copper-less” click chemistry reaction. Bertozzi et al., Compositions and methods for modification of biomolecules, U.S. Patent App. No. 20060110782.

For example, microbes may be first treated with an effective amount of an azide modified nucleoside analog, for example, AzdU, for a defined period of time such that the azide-modified nucleoside analog is incorporated into newly synthesized microbial DNA. After the addition of AzdU, cells are treated with an effective amount of a compound or molecule with a reactive cycloalkyne moiety such that a strained [3+2] cycloaddition reaction occurs between the azide and cycloalkyne moieties. The cycloalkyne may be modified to further comprise a dye label, which may then be measured using standard methods, including but not limited to, flow cytometry, fluorescence microscopy, imaging, multi-well plate assays, or high content screening; a biotin label that can be used with a pulldown reagent; an HRP enzyme; etc. Cycloalkynes that may be used in strained [3+2] cycloaddition reactions in order to label DNA include, but are not limited to: cyclooctynes, difluorocyclooctynes, heterocycloalkynes, dichlorocyclooctynes, dibromocyclooctynes, or diiodocyclooctynes. Other chemistries known in the art may be applied to the labeling of microbial DNA. For example, azide-phosphine chemistry described by Bertozzi et al., also known as the Staudinger ligation, may be used to detect incorporation of an azide-modified nucleoside analog, e.g. AzdU, into newly synthesized microbial DNA. See Bertozzi et al., Chemoselective ligation, U.S. Patent App. No. 20070037964. Microbes are first contacted with an effective amount of an azide-modified nucleoside analog, e.g. AzdU, for a defined period of time. Then, microbes are reacted with an engineered phosphine moiety. One example of an engineered phosphine moiety is 2-diphenylphosphanyl-benzoic acid methyl ester. When azide-phosphine chemistry is used to label microbial DNA, the engineered phosphine moiety further comprises a dye molecule, a biotin moiety, an enzyme, etc. Once the reaction between the azide and phosphine moieties has taken place, the biotin molecule can be used in a pulldown assay; etc.

To measure both baseline microbial proliferation and a subsequent change in microbial proliferation, the disclosure further provides for the use of a second nucleoside or nucleotide analog that is differentially labeled than the first used nucleoside or nucleotide analog. It is further envisioned that a third and/or a fourth nucleoside or nucleotide analog could be used, so as to measure the effectiveness of an antimicrobial (e.g., antibiotic) on microbial proliferation or gene expression by the microorganism. A baseline synthesis rate can be recorded by the labeling of the nucleic acid with a first nucleoside or nucleotide analog. There is no need to remove the first nucleoside or nucleotide analog, prior to the introduction of the second nucleoside or nucleotide analog. Further, by first removing the first nucleoside or nucleotide analog prior to the introduction of the second nucleoside or nucleotide analog may make an accurate determination of microbial proliferation rate difficult. In addition, the no wash step makes the process compatible with high throughput screening (HTS).

One of the main advantages of the compositions, methods and kits disclosed herein is that the identification of microorganism(s) in a sample does not need a long culturing step, unlike standard protocols. As DNA is constantly being produced in viable, proliferating organisms, the compositions, methods and kits of the disclosure can identify the microorganism without any need to use a culturing step to grow up the microorganisms. Instead, the compositions, methods and kits of the disclosure utilize an incubation step where a sample is incubated in the presence of one or more types of nucleoside or nucleotide analogs for a minimal period of time such that the nucleoside or nucleotide analogs are incorporated into nascently made microbial nucleic acids. Accordingly, the sample once obtained can be incubated in the presence of one or more types of nucleoside or nucleotide analogs for around 5 min, 10 min, 15 min, 20 min, 25 min, 30 min, 35 min, 40 min, 45 min, 50 min, 55 min, 60 min, 65 min, 70 min, 75 min, 80 min, 85 min, 90 min, 95 min, 100 min, 105 min, 110 min, 115 min, 120 min, 125 min, 130 min, 145 min, 150 min, 155 min, 160 min, 165 min, 170 min, 175 min, 180 min, 190 min, 200 min, 220 min, 330 min, 240 min, 260 min, 280 min, 300 min, 350 min, 400 min, 500, min, 600 min, or any range that includes or is between any two of the foregoing time points, including factional increments thereof.

The disclosure further provides for labeling the nascently made microbial nucleic acids containing the nucleoside or nucleotide analogs with one or more types of labeling reagents. The labeling reagents disclosed herein bind with specificity to the nucleoside or nucleotide analogs. For example, the labeling reagent can be a first antibody, which may be conjugated to a label or bound by a second antibody that is covalently attached to a label, wherein the first antibody binds to the incorporated nucleoside or nucleotide analog. Examples of such first antibodies, can include anti-BrdU antibodies, anti-ldU antibodies, and anti-CldU antibodies, all of which are commercially available from various vendors. However, other antibodies which could selectively bind to incorporated nucleoside or nucleotide analogs (as described above) are also envisioned. In regards to the second antibody, the second antibody can be bound to a substrate, such as beads or a plate. Therefore, the second antibody can function as a pulldown reagent that allows for the isolation or purification of newly synthesized microbial nucleic acids. Alternatively, the labeling reagent can be compounds that comprise functional groups (e.g., azides) which are designed so that they can undergo a chemical reaction with nucleoside or nucleotide analogs that have complementary bioorthogonal functional groups (e.g., alkynyl groups), and which comprise a label, such as a dye moiety, a fluorophore moiety, an affinity ligand (e.g., GST, biotin, histidine, etc.), enzyme (e.g., horse radish peroxidase), and the like. Examples of labeling reagents comprising a biotin label include the following:

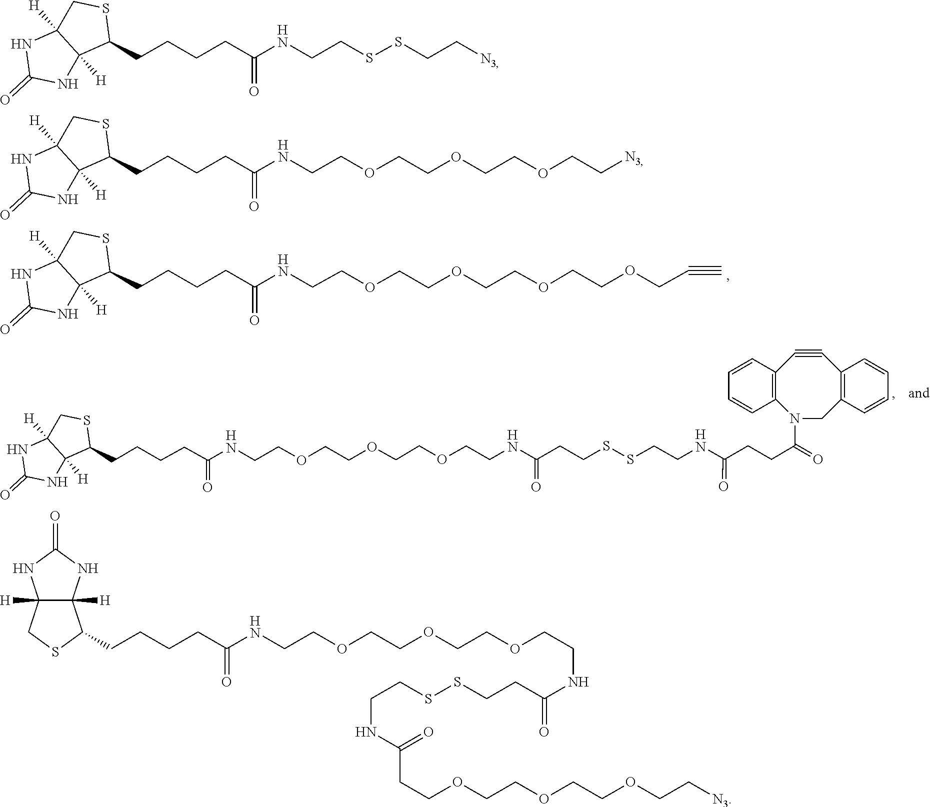

As already mentioned above, the role of a label is to allow visualization or detection of a nucleic acid polymer, e.g., newly synthesized microbial DNA, following labeling. Typically, a label (or detectable agent or moiety) is selected such that it can be selectively bound by a pulldown reagent, or alternatively can generate a signal which can be measured and whose intensity is related (e.g., proportional) to the amount of labeled nucleic acid polymer, e.g., in a sample being analyzed. Accordingly, it is envisaged that multiple labels can be used to detect, identify and quantitate newly synthesized microbial nucleic acids, e.g., a first label can be bound by a pulldown reagent to provide for isolation of the newly synthesized microbial nucleic acids, and a second, third or more labels can be used generate signals that are measured and whose intensity is related to the amount of labeled nucleic acid polymer in a sample being analyzed. Such uses of multiple labels are especially advantageous for determining the rate of proliferation or new nucleic acid synthesis; or testing the effect of an administered agent, such as antibiotics. Further, the labeling reagents can further comprise a chemically cleavable linker or enzymatically cleavable linker, so that the label can be removed if so needed. Any number of chemically cleavable linkers can be used, but generally should be linker that can cleaved under mild reaction conditions, such as acid-labile-based linkers, base-labile-based linkers, diazo-based linkers or disulfide-based linkers. Examples of acid-labile based linkers include linkers comprising hydrazone, enamine, enol ether, imine or cis-aconityl groups. Examples of base-labile based linkers include carbamate-based and ester-based linkers. Alternatively, the cleavable linker can be an enzymatically cleavable linker. Examples of enzymatically cleavable linkers include peptide-based linkers or β-glucuronide-based linkers.

The method for the identification and analysis of microorganisms in a sample as described herein further provides for the isolation or purification of labeled microbial nucleic acids. In a particular embodiment, labeled microbial nucleic acids can be purified or isolated using a pulldown reagent. In such a case, the newly synthesized microbial nucleic acids are labeled with a labeling reagent that binds to or with an incorporated nucleoside analog as is described herein, and which comprises a label which can be selectively bound by an immobilized pulldown regent. The labeling reagent can be an antibody or another type of an affinity-based ligand (e.g., GST, biotin, histidine, etc.). The pulldown reagent can be a second antibody specific for the labeling reagent, or can be another type of agent or compound that has high and selective affinity for the labeling reagent. For example, the labeling reagent can be biotin-based molecule that binds with the nucleoside analog via click chemistry, and which can itself be selectively bound by a pulldown reagent comprising avidin or streptavidin. Thus, the interaction between the biotin-based labeling reagent and the strepavidin-based pulldown agent allows for the isolation or purification of ‘labeled’ microbial nucleic acids from ‘unlabeled’ microbial nucleic acids, and other microbial constituents. Typically, the pulldown reagent is immobilized onto a solid support, such as a plate, beads, nano- or micromaterials (e.g., magnetic nanoparticles).

The disclosure also provides that the compositions, methods, and kits of disclosure can detect the effectiveness of an agent on microbial viability, growth and proliferation. For example, an antimicrobial agent can be added directly to the sample and the resulting effect on microbial viability, growth and/or proliferation can be determined based upon the detection, or lack thereof, of newly synthesized nucleic acids using the methods of the disclosure. For more controlled results, the sample can be split into two samples, a ‘control’ sample and a ‘treated’ sample, whereby the antimicrobial agent is added to the ‘treated’ sample and a vehicle control is added to ‘control sample,’ and determining any differences in the production of newly synthesized microbial nucleic acids between the two samples, whereby if the ‘treated’ sample has less or no newly synthesized microbial nucleic acids in comparison to the ‘control’ sample would be indicative of the effectiveness of the antimicrobial agent. Alternatively, the effect of the antimicrobial agent can be determined in the system to be tested, based upon taking a first sample prior to administration of the antimicrobial agent, and taking a second, third, or more samples at one or more time points post-administration of the antimicrobial agent. For example, a blood sample can be obtained from a sepsis human patient prior to and after administration of antibiotics, whereby decreased rate or absence of newly synthesized nucleic acids is indicative of the effectiveness of the antibiotics on the viability, growth and/or proliferation of the bacteria causing sepsis. Examples of antimicrobial agents that can be used with the compositions, methods and kits of the disclosure, include, but are not limited to, antibiotics, antifungals, antivirals, and antiparasitics. Antibiotics are a type of antimicrobial substance active against bacteria and is the most important type of antibacterial agent for fighting bacterial infections. Antibiotic medications are widely used in the treatment and prevention of such infections. They may either kill or inhibit the growth of bacteria. Examples of antibiotics that can be used with the compositions, methods, and kits disclosed herein include, but are not limited to, penicillins, such as amoxicillin, ampicillin, bacampicillin, carbenicillin, cloxacillin, dicloxacillin, flucloxacillin, mezlocillin, nafcillin, oxacillin, penicillin G, penicillin V, piperacillin, pivampicillin, pivmecillinam, and ticarcillin; cephalosporins, such as cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cefcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, cefoperazone, ceftazidime, cefclidine, cefepime, cefluprenam, cefoselis, cefozopran, cefpirome, cefquinome, ceftobiprole, ceftaroline, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, cefuracetime, and ceftioxide; monobactams, such as aztreonam; carbapenems, such as imipenem, doripenem, ertapenem, and meropenem; macrolide antibiotics, such as azithromycin, erythromycin, clarithromycin, dirithromycin, roxithromycin, and telithromycin; lincosamides, such as clindamycin and lincomycin; aminoglycoside antibiotics, such as amikacin, gentamicin, kanamycin, neomycin, netilmicin, paromomycin, streptomycin, and tobramycin; quinolone antibiotics, such as flumequine, nalidixic acid, oxolinic acid, piromidic acid, pipemidic acid, rosoxacin, ciprofloxacin, enoxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, besifloxacin, delafloxacin, clinafloxacin, gemifloxacin, prulifloxacin, sitafloxacin, and trovafloxacin; sulfonamides, such as sulfamethizole, sulfamethoxazole, sulfisoxazole, trimethoprim-sulfamethoxazole; tetracycline antibiotics, such as demeclocycline, doxycycline, minocycline, oxytetracycline, tetracycline, and tigecycline; glycopeptide antibiotics such as vancomycin and teicoplanin; lipoglycopeptide antibiotics, such as telavancin; oxazolidinone antibiotics, such as linezolid, and cycloserine; rifamycins such as rifampin, rifabutin, rifapentine, and rifalazil; tuberactinomycins such as viomycin and capreomycin; other antibiotics, such as bacitracin, polymyxin B, chloramphenicol, metronidazole, tinidazole, and nitrofurantoin. Antifungals are a type of antimicrobial substance active against fungi and is the most important type of antifungal agent for fighting fungal infections. Antifungal medications are widely used in the treatment and prevention of such infections. They may either kill or inhibit the growth of fungi. Examples of antifungals that can be used with the compositions, methods, and kits disclosed herein include, but are not limited to, allylamine antifungals such as amorolfine, butenafine, naftifine, and terbinafine; imidazole antifungals such as, bifonazole, butoconazole, clotrimazole, econazole, fenticonazole, ketoconazole, isoconazole, luliconazole, miconazole, omoconazole, oxiconazole, sertaconazole, sulconazole, tioconazole, and terconazole; triazole antifungals such as albaconazole, efinaconazole, fluconazole, isavuconazole, itraconazole, posaconazole, ravuconazole, terconazole, and voriconazole; and thiazole antifungals, such as abafungin; polyene antifungals such as amphotericin B, nystatin, natamycin, and trichomycin; echinocandins, such as anidulafungin, caspofungin, and micafungin; thiocarbamate antifungals, such as tolnaftate; antimetabolite antifungals, such as flucytosine; benzylamines, such as butenafine; other antifungals, such as griseofulvin, ciclopirox, selenium sulfide, and tavaborole. Antivirals are medications that prevent the entry, replication, spread, and/or maturation of viruses. Examples of antivirals that can be used with the compositions, methods, and kits disclosed herein include, but are not limited to, anti-herpetic agents such as acyclovir, brivudine, docosanol, famciclovir, foscarnet, idoxuridine, penciclovir, trifluridine, vidarabine, cytarabine, valacyclovir, tromatandine, and pritelivir; anti-influenza agents, such as amantadine, rimantadine, oseltamivir, peramivir, and zanamivir; NS3/4A protease inhibitors, such as asunaprevir, boceprevir, ciluprevir, danoprevir, faldaprevir, glecaprevir, grazoprevir, narlaprevir, paritaprevir, simeprevir, sovaprevir, telaprevir, vaniprevir, vedroprevir, and voxilaprevir; NS5A inhibitors, such as daclatasvir, elbasvir, ledipasvir, odalasvir, ombitasvir, pibrentasvir, ravidasvir, ruzasvir, samatasvir, and velpatasvir; NS5B RNA polymerase inhibitors, such as beclabuvir, dasabuvir, deleobuvir, filibuvir, setrobuvir, sofosbuvir, radalbuvir, and uprifosbuvir; anti-hepatitis B, such as lamivudine, telbivudine, clevudine, adefovir, tenofvir disoproxil, and tenofovir alafenamide; entry/fusion inhibitors, such as enfuvirtide, maraviroc, vicriviroc, cenicriviroc, PRO 140, ibalizumab, and fostemsavir; reverse transcriptase inhibitors, such as didanosine, emtricitabine, lamivudine, stavudine, zidovudine, amdoxovir, apricitabine, censavudine, elvucitabine, racivir, stampidine, 4′-ethynyl-2-fluoro-2′-deoxyadenosine, zalcitabine, efavirenz, nevirapine, delavirdine, etravirine, rilpivirine, and doravirine; inegrase inhibitors, such as dolutegravir, elvitegravir, raltegravir, BI 224436, cabotegravir, bictegravir, and MK-2048; maturation inhibitors, such as bevirimat, and BMS-955176; protease inhibitors, such as, amprenavir, fosamprenavir, indinavir, lopinavir, nelfinavir, ritonavir, saquinavir, atazanavir, darunavir, and tipranavir; integrase inhibitors, such as dolutegravir, elvitegravir, raltegravir, BI 224436, cabotegravir, bictegravir, and MK-2048; Nucleotide analogues/NtRTIs such as tenofovir disoproxil, tenofovir alafenamide (TAF); pharmacokinetic boosters, such as cobicistat and ritonavir; and interferons, such as interferon-α, and peginterferon-α; other antivirals, such as methisazone, rifampicin, imiquimod, resiquimod, podophyllotoxin, fomivirsen, cidofovir, pleconaril, favipiravir, galidesivir, remdesivir, mericitabine, MK-608, NITD008, moroxydine, tromantadine and triazavirin.

The compositions, methods and kits disclosed herein also provides for the identification of microorganisms in a sample by identifying newly synthesized microbial nucleic acids by use of a microarray that comprises probes to nucleic acids from many different microorganisms. Typically, the microarray will have probes to nucleic acids from 20, 25, 30, 35, 40, 45, 50, 55, 60, 65, 70, 75, 80, 85, 90, 95, 100, 110, 120, 130, 140, 150, 160, 180, 190, 200, 300, 400, 500, 600, 700, 800, 900, 1000, 5000, 10000, 15000, 20000, 30000, 40000, 50000, 100000 or any range that includes or is between any two of the foregoing values, including factional increments thereof, different microorganisms. The probes are typically designed to have sequences complementary to segments of one or more target organism genomes (e.g., 16S rRNA). Oligos may be spotted onto the array by mechanical deposition, sprayed on with a modified inkjet printer head or synthesized in situ through a series of photocatalyzed reactions. Probes are placed on the array in a rectangular grid of ‘features’, each containing many copies of the same oligo. The density of features on the array varies between platforms, from 20,000 spots per slide for a typical spotted array, to several million for platforms such as NimbleGen and Affymetrix that use in situ synthesized oligos. Arrays may be subdivided with a gasket into subarrays, allowing multiple samples to be tested on one slide. Replicate features, scattered randomly across the array, may be used to allow correction for scratches and spatial effects. On some arrays, negative control probes with random sequences are included, to provide a threshold level for background noise correction. Any number of pathogen detection microarrays have been described in the art, including ViroChip (Wang D. et al., PLoS Biol. 2003; 1:E2); resequencing pathogen microarrays (Leski T. et al. PLoS ONE. 2009; 4:e6569); universal detection microarray (Belosludtsev Y. et al., BioTechniques. 2004; 37:654-8, 66); GreeneChip (Quan et al., J Clin Microbiol. 2007; 45:2359-64); and Lawrence Livermore microbial detection array (Gardner S. et al. BMC Genomics. 2010; 11:668). For example, the Lawrence Livermore microbial detection array has probes for nearly 6,000 viruses and 15,000 bacteria as well as fungi and protozoa organisms. After applying the newly synthesized microbial DNA to the microarray, any probe that detects its specific sequence will fluoresce, and be read by a scanner. The raw data from the scanner is then analyzed using algorithms. Bioinformatics is used in identifying the large numbers of nucleic acid sequences, or probes, which are the signatures of microbes.

The compositions, methods and kits disclosed herein also provides for the identification of microorganisms in a sample by sequencing newly synthesized microbial nucleic acids that have incorporated nucleoside or nucleotide analogs of disclosure. Any number of sequencing methodologies may be used to sequence newly synthesized microbial DNA, or microbial RNA that has been reverse transcribed into cDNA, including sequencing technologies based upon the Sanger dideoxy chain termination sequencing method, in vitro transposition, next-generation sequence platforms including the 454 FLX™ or 454 TITANIUM™ (Roche), the SOLEXA™ Genome Analyzer (Illumina), the HELISCOPE™ Single Molecule Sequencer (Helicos Biosciences), and the SOLID™ DNA Sequencer (Life Technologies/Applied Biosystems) instruments), as well as other platforms still under development by companies such as Intelligent Biosystems and Pacific Biosystems. Although the chemistry by which sequence information is generated varies for the different next-generation sequencing platforms, all of them share the common feature of generating sequence data from a very large number of sequencing templates, on which the sequencing reactions are run simultaneously. In general, the data from all of these sequencing reactions are collected using a scanner, and then assembled and analyzed using computers and powerful bioinformatics programs. The sequencing reactions are performed, read, assembled, and analyzed in a “massively parallel” or “multiplex” fashion. The massively parallel nature of these instruments has required a change in thinking about what kind of sequencing templates are needed and how to generate them in order to obtain the maximum possible amounts of sequencing data from these powerful instruments. Thus, rather than requiring genomic libraries of DNA clones in E. coli, it is now important to think in terms of in vitro systems for generating DNA fragment libraries comprising a collection or population of DNA fragments generated from target DNA in a sample, wherein the combination of all of the DNA fragments in the collection or population exhibits sequences that are qualitatively and/or quantitatively representative of the sequence of the target DNA from which the DNA fragments were generated. In fact, in some cases, it is necessary to think in terms of generating DNA fragment libraries consisting of multiple genomic DNA fragment libraries, each of which is labeled with a different address tag or bar code to permit identification of the source of each fragment sequenced.

In general, these next-generation sequencing methods require fragmentation of genomic DNA or double-stranded cDNA (prepared from RNA) into smaller ssDNA fragments and addition of tags to at least one strand or preferably both strands of the ssDNA fragments. In some methods, the tags provide priming sites for DNA sequencing using a DNA polymerase. In some methods, the tags also provide sites for capturing the fragments onto a surface, such as a bead (e.g., prior to emulsion PCR amplification for some of these methods; e.g., using methods as described in U.S. Pat. No. 7,323,305). In most cases, the DNA fragment libraries used as templates for next-generation sequencing comprise 5′- and 3′-tagged DNA fragments or “di-tagged DNA fragments.” In general, current methods for generating DNA fragment libraries for next-generation sequencing comprise fragmenting the target DNA that one desires to sequence (e.g., target DNA comprising genomic DNA or double-stranded cDNA after reverse transcription of RNA) using a sonicator, nebulizer, or a nuclease, and joining (e.g., by ligation) oligonucleotides consisting of adapters or tags to the 5′ and 3′ ends of the fragments. Some of the next-generation sequencing methods use circular ssDNA substrates in their sequencing process. For example, U.S. Patent Application Nos. 20090011943; 20090005252; 20080318796; 20080234136; 20080213771; 20070099208; and 20070072208 of Drmanac et al. disclose generation of circular ssDNA templates for massively parallel DNA sequencing. U.S. Patent Application No. 20080242560 of Gunderson and Steemers discloses methods comprising: making digital DNA balls (see, e.g., FIG. 8 in U.S. Patent Application No. 20080242560); and/or locus-specific cleavage and amplification of DNA, such as genomic DNA, including for amplification by multiple displacement amplification or whole genome amplification (e.g., FIG. 17 therein) or by hyperbranched RCA (e.g., FIG. 18 therein) for generating amplified nucleic acid arrays (e.g., ILLUMINA BeadArrays™; ILLUMINA, San Diego Calif., USA).

In a particular embodiment, the disclosure provides for the use of transposome-based sequencing methods to identify microorganisms in the sample. Such transposome-based sequencing methods are described in US2014/0162897; US2015/0368638; US2018/0245069; US2018/0023119; WO20122103545; WO20150160895; WO2016130704; WO2019028047; U.S. Pat. No. 9,574,226; EP3161152; the disclosures of which are incorporated in their entirety for this disclosure. The number of steps required to transform a target nucleic acid such as DNA into adaptor-modified templates ready for next generation sequencing can be minimized by the use of transposase-mediated fragmentation and tagging. This process, referred to herein as “tagmentation,” often involves modification of a target nucleic acid by a transposome complex comprising a transposase enzyme complexed with a transposon pair comprising a single-stranded adaptor sequence and a double-stranded transposon end sequence region, along with optional additional sequences designed for a particular purpose. Tagmentation results in the simultaneous fragmentation of the target nucleic acid and ligation of the adaptors to the 5′ ends of both strands of duplex nucleic acid fragments. Where the transposome complexes are support-bound, the resulting fragments are bound to the solid support following the tagmentation reaction (either directly in the case of the 5′ linked transposome complexes, or via hybridization in the case of the 3′ linked transposome complexes). In particular, by using transposase and a transposon end compositions described herein one can generate libraries of di-tagged linear ssDNA fragments or tagged circular ssDNA fragments (and amplification products thereof) from target microbial DNA (including double-stranded cDNA prepared from microbial RNA) for genomic, subgenomic, transcriptomic, or metagenomic analysis or analysis of microbial RNA expression (e.g., for use in making labeled target for microarray analysis; e.g., for analysis of copy number variation, for detection and analysis of single nucleotide polymorphisms, and for finding genes from environmental samples such as soil or water sources).

In a particular embodiment, the transposome-based sequencing method described herein uses an in vitro transposition reaction to simultaneously break newly synthesized microbial DNA into fragments and join a tag to the 5′-end of each fragment. The in vitro transposition reaction can be performed by assembling the reaction using either separate transposase and transposon end compositions or a single transposome composition comprising a stable complex formed between the transposase and the transposon end composition. Therefore, it will be understood that any transposome-based sequencing method that describes the use of a transposase and a transposon end composition could also use a transposome composition made from the transposase and the transposon end composition, and any transposome-based sequencing method that describes the use of a transposome composition could also use the separate transposase and a transposon end compositions of which the transposome composition is composed.

The transposome-based sequencing method described herein can be used to generate a library of tagged DNA fragments from newly synthesized microbial DNA, the transposome-based sequencing method comprising: incubating the newly synthesized microbial DNA in an in vitro transposition reaction with at least one transposase and a transposon end composition with which the transposase forms a transposition complex, the transposon end composition comprising (i) a transferred strand that exhibits a transferred transposon end sequence and, optionally, an additional sequence 5′- of the transferred transposon end sequence, and (ii) a non-transferred strand that exhibits a sequence that is complementary to the transferred transposon end sequence, under conditions and for sufficient time wherein multiple insertions into the newly synthesized microbial DNA can occur, each of which results in joining of a first tag comprising or consisting of the transferred strand to the 5′ end of a nucleotide in the target DNA, thereby fragmenting the target DNA and generating a population of annealed 5′-tagged DNA fragments, each of which has the first tag on the 5′-end; and then joining the 3′-ends of the 5′-tagged DNA fragments to the first tag or to a second tag, thereby generating a library of tagged DNA fragments (e.g., comprising either tagged circular ssDNA fragments or 5′- and 3′-tagged DNA fragments (or “di-tagged DNA fragments”)). In a further embodiment, the transposome-based sequencing method described above uses separate transposase and transposon end compositions, whereas in other embodiments, the transposome-based sequencing method is performed using a transposome composition comprising the complex formed between the transposase and the transposon end composition.

The disclosure further provides for the identification of microorganisms by using transposome-based sequencing method where the transposome complexes bound to a solid support. An example of a commercial product using bead-linked transposomes for sequencing is the Nextera™ DNA Flex system provided by Illumina®. The Nextera™ DNA Flex system can be used for the identification of microorganisms using the methods described herein. Nucleic acid fragment libraries may be prepared using a transposome-based method where two transposon end sequences, one linked to a tag sequence, and a transposase form a transposome complex. The transposome complexes are used to fragment and tag target nucleic acids in solution to generate a sequencer-ready tagmented library. The transposome complexes may be immobilized on a solid surface, such as through a biotin appended at the 5′ end of one of the two end sequences. Use of immobilized transposomes provides significant advantages over solution-phase approaches by reducing hands-on and overall library preparation time, cost, and reagent requirements, lowering sample input requirements, and enabling the use of unpurified or degraded samples as a starting point for library preparation. Exemplary transposition procedures and systems for immobilization of transposomes on a solid surface to result in uniform fragment size and library yield are described in detail in WO2014/108810 and WO2016/189331, each of which is incorporated herein by reference in its entirety. In certain bead-based tagmentation methods described in PCT Publ. No. WO2016/189331 and US 2014/093916A1, transposomes are bound to magnetic beads using biotin-streptavidin interactions.

Generally, a transposome is immobilized on a substrate, such as a slide or bead, using covalent or non-covalent binding partners, e.g., an affinity element and an affinity binding partner. For example, a transposome complex is immobilized on a streptavidin-coated bead through a biotinylated linker attached to the transposome complex. The newly synthesized microbial nucleic acids are captured by the immobilized transposome complex and the nucleic acids are fragmented and tagged (“tagmentation”). The tagged fragments are amplified, amplicons of interest are optionally captured (e.g., via hybridization probes), and the tagged fragments are sequenced.

Using solid support-linked transposome complexes for library preparation reduces the need for normalization of sample input going into the library preparation process and for normalization of library output before enrichment or sequencing steps. Using these complexes also produces libraries with more consistent insert sizes relative to solution-phase methods, even when varying sample input concentrations are used. In some embodiments, the transposome complexes are immobilized to a support via one or more polynucleotides (e.g., oligonucleotides), such as a polynucleotide (oligonucleotide) comprising a transposon end sequence. In some embodiments, the transposome complex may be immobilized via a linker appended to the end of a transposon sequence, for example, coupling the transposase enzyme to the solid support. In some embodiments, both the transposase enzyme and the transposon polynucleotide (e.g., oligonucleotide) are immobilized to the solid support. When referring to immobilization of molecules (e.g., nucleic acids, enzymes) to a solid support, the terms “immobilized”, “affixed” and “attached” are used interchangeably herein and both terms are intended to encompass direct or indirect, covalent or non-covalent attachment, unless indicated otherwise, either explicitly or by context. In certain embodiments of the present disclosure covalent attachment may be preferred, but generally all that is required is that the molecules (e.g. nucleic acids, enzymes) remain immobilized or attached to the support under the conditions in which it is intended to use the support, for example in applications requiring nucleic acid amplification and/or sequencing. In some instances, in bead based tagmentation, transposomes may be bound to a bead surface via a ligand pair, e.g., an affinity element and affinity binding partner.

Transposon based technology can be utilized for fragmenting DNA, for example, as exemplified in the workflow for NEXTERA™ XT and FLEX DNA sample preparation kits (Illumina, Inc.), wherein newly synthesized microbial nucleic acids are treated with transposome complexes that simultaneously fragment and tag (“tagmentation”) the target, thereby creating a population of fragmented nucleic acid molecules tagged with unique adaptor sequences at the ends of the fragments.

A transposition reaction is a reaction wherein one or more transposons are inserted into target nucleic acids at random sites or almost random sites. Components in a transposition reaction include a transposase (or other enzyme capable of fragmenting and tagging a nucleic acid as described herein, such as an integrase) and a transposon element that includes a double-stranded transposon end sequence that binds to the enzyme, and an adaptor sequence attached to one of the two transposon end sequences. One strand of the double-stranded transposon end sequence is transferred to one strand of the target nucleic acid and the complementary transposon end sequence strand is not (i.e., a non-transferred transposon sequence). The adaptor sequence can comprise one or more functional sequences (e.g., primer sequences) as needed or desired.

Thus, in a further embodiment, the identification and analysis of microorganisms in a sample further comprises the steps of generating a library of tagged nucleic acid fragments, comprising: providing a solid support comprising a transposome complex described herein immobilized thereon; and contacting the solid support with isolated or purified newly synthesized microbial nucleic acids under conditions sufficient to fragment the target nucleic acid into a plurality of target fragments, and to join the 3′ end of the first transposon to the 5′ ends of the target fragments to provide a plurality of 5′ tagged target fragments. In a further embodiment, the method further comprises amplifying the 5′ tagged target fragments. In yet a further embodiment, the methods further comprise sequencing one or more of the 5′ tagged target fragments or amplification products thereof. In some aspects, the disclosure provides for a library of 5′ tagged fragments of the newly synthesized microbial nucleic acids produced by the methods described herein.

In another aspect, the present invention provides kits that includes at least one nucleoside analog and labeling reagent of the invention. The kit will generally also include instructions for using the nucleoside analog and labeling reagent in one or more methods, typically for detecting or measuring a change in microbial nucleic acid synthesis.

In an exemplary embodiment, the kit includes a nucleoside or nucleotide analog that contains a bioorthogonal functional moiety, and a first labeling reagent that can undergo click chemistry with the bioorthogonal functional moiety. Additional kit components include pulldown reagents, buffers, other detection reagents and standards.

The following examples are intended to illustrate but not limit the disclosure. While they are typical of those that might be used, other procedures known to those skilled in the art may alternatively be used.

### EXAMPLES

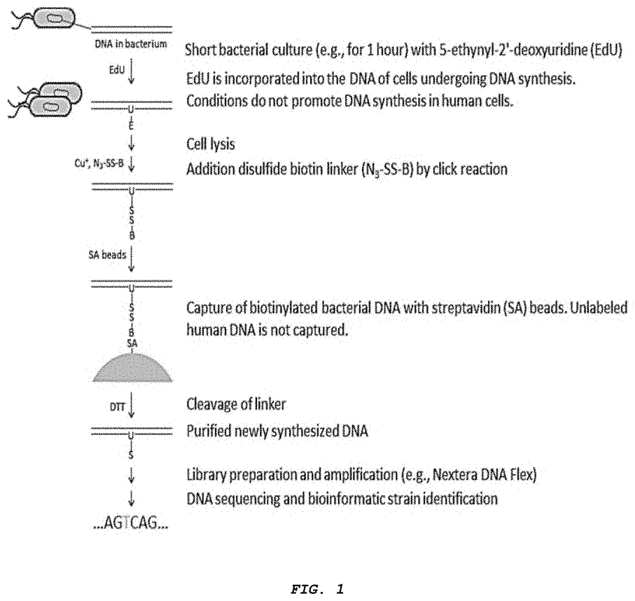

Exemplary method to detect and identify bacteria in a sample from a sepsis patient. The methodologies and technologies of the disclosure allow for detection of bacteria in a sepsis patient. As shown in FIG. 1, a blood sample is cultured in a medium containing a nucleoside labeling reagent 5-ethynyl-2′-deoxyuridine (EdU). After a short period of culture (minutes to a few hours), live cells undergoing DNA synthesis incorporate the EdU into their genomes. Here, if antibiotic resistance is to be queried, antibiotics of interest can be included in the culture medium. After rapid culturing, the cells are then lysed and then optionally DNA can be purified from the lysate. The newly synthesized DNA. containing EdU is then labeled with biotin via a click reaction with an azide-disulfide-biotin linker. Having been labeled with biotin, the newly synthesized DNA is then captured by streptavidin-conjugated beads. After washing the beads, the DNA is freed from the beads by addition of dithiothreitol (DTT). To identify the bacterium that produced the DNA, sequencing libraries are prepared using standard methods (e.g., Illumina Nextera DNA Flex with PCR library amplification) and then sequenced. Bioinformatic analysis of the sequences reveals the identity of the sepsis-causing bacteria.

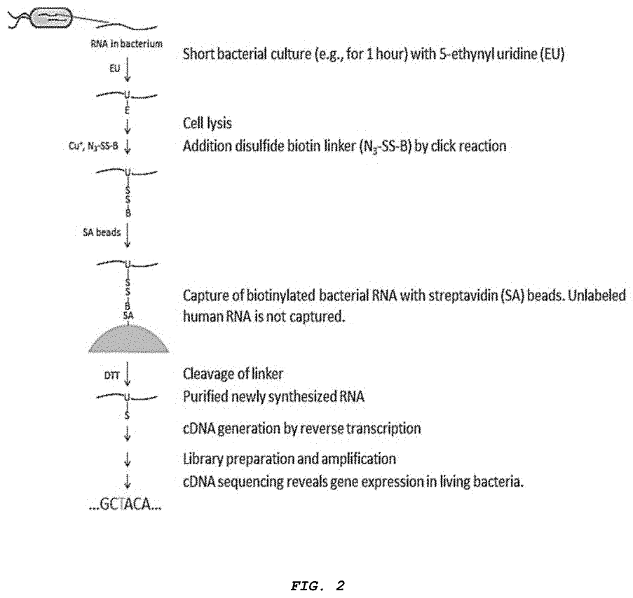

Exemplary method to rapidly analyze microbial gene expression in samples containing living microorganisms. The methodologies and technologies of the disclosure can also be used to rapidly analyze microbial gene expression in samples containing living microorganisms (FIG. 2). Instead of culturing with EdU, samples are cultured with 5-ethynyl-uridine (EU), which is then incorporated into the newly synthesized microbial RNA. After biotin labeling and streptavidin-based purification of the RNA, the RNA is reverse transcribed into cDNA. From the cDNA, sequencing libraries are prepared. Sequencing and bioinformatic analysis then reveal the gene expression of the living microbes in the sample. This gene expression analysis could be used to identify the genes causing a disease phenotype or determine whether the microorganism is responding to antibiotics. In addition to information about gene expression, RNA sequence analysis can also be used to identify strains. Because RNA is synthesized in living, non-proliferating microbes, RNA analysis could be able to identify contaminating or infectious microbes that are viable but not replicating in culture.

It will be understood that various modifications may be made without departing from the spirit and scope of this disclosure. Accordingly, other embodiments are within the scope of the following claims.

1. A method for the identification and analysis of viable and/or proliferating microorganisms in a sample, comprising:
(a) obtaining a sample having or suspected of having one or more types of microorganisms;
(b) incubating the sample in the presence of one or more types of nucleoside or nucleotide analogs, wherein the one or more types of nucleoside or nucleotide analogs are incorporated into newly synthesized microbial nucleic acids, the one or more being selected from the croup consisting of 2-ethynyl-adenosine, N6-propargyl-adenosine, 2′-(O-propargyl)-adenosine, 3′-(O-propargyl)-adenosine, 5-ethynyl-cytidine, 5-ethynyl-2′-deoxycytidine, 2′-(O-propargyl)-cytidine, 3′-(O-propargyl)-cytidine, 2′-(O-propargyl)-guanosine, 3′-(O-propargyl)-guanosine, 5-ethynyl-uridine, 5-ethynyl-2′-deoxyuridine, 2′-(O-propargyl)-uridine, 3′-(O-propargyl)-uridine, (2'S)-2′-deoxy-2′-fluoro-5-ethynyluridine, (2'S)-2′-fluoro-5-ethynyluridine, 2′(S)-2′-deoxy-2′-fluoro-5-ethynyluridine, (2'S)-2′-fluoro-5-ethynyluridine, 8-azido-adenosine, N⁶-(6-azido)hexyl-2′deoxy-adenosine, 2′-azido-2′-deoxyadenosine, 5-azidomethyl-uridine, 5-(15-azido-4,7,10,13-tetraoxa-pentadecanoyl-aminoallyl)-2′-deoxyuridine, 5-(3-azidopropyl)-uridine, 5-azido-PEG₄-uridine, 5-azido-PEG₄-cytidine, 5-azido-PEG₄-2′-deoxycytidine, 5-bromo-2′deoxyuridine, 5-bromouridine, 5-iodo-2′deoxyuridine, 5-iodouridine, and any combination thereof;
(c) labelling newly synthesized microbial nucleic acids by contacting the newly synthesized microbial nucleic acids with a labelling reagent that selectively binds to or with the one or more types of nucleoside or nucleotide analogs;
(d) isolating or purifying the labelled newly synthesized microbial nucleic acids; and
(e) determining the identity of the viable and/or proliferating microorganisms in the sample based upon sequencing or determining the identity of the isolated or purified newly synthesized microbial nucleic acids.

2. The method of claim 1, wherein the sample is obtained from a subject suspected of having or having a microbial infection.

3. (canceled)

4. The method of claim 1, wherein for (a), the obtained sample is processed using a dehosting method prior to (b) in order to selectively remove nonmicrobial nucleic acids.

5. The method of claim 4, wherein the dehosting method comprises:
removing nonmicrobial nucleic acids by:
(a) selectively cleaving nonmicrobial DNA by contacting the obtained sample with a recombinant protein comprising: a binding domain that selectively binds to nonmicrobial nucleic acids bound by histone(s) or to nonmicrobial nucleic acids comprising methylated CpG residues, and a nuclease domain having activity to cleave nucleic acids; or
(b) use of an affinity agent that is bound to a solid substrate that selectively binds to nucleic acids bound by histone(s) or selectively binds to methylated CpG residues of nonmicrobial nucleic acids.

6. The method of claim 1, wherein the sample is an environmental sample obtained from an environmental test site.

7. The method of claim 6, wherein the environmental site is being tested for microbial contamination.

8. The method of claim 1, wherein the sample is a sample obtained from a foodstuff suspected of microbial contamination.

9. The method of claim 1, wherein the one or more types of microorganisms are bacteria, fungi, viruses, algae, archaea, and/or protozoa.

10-16. (canceled)

17. The method of claim 1, wherein the sample is incubated in the presence of one or more types of nucleoside or nucleotide analogs for 5 min to 180 min.

18. (canceled)

19. The method of claim 1, wherein the labeling reagent is an antibody that binds with high specificity to the one or more types of nucleoside or nucleotide analogs.

20. The method of claim 19, wherein the antibody binds with high specificity to 5-bromo-2′deoxyuridine, or iododeoxyuridine.

21. The method of claim 1, wherein the labelling reagent binds to or with the one or more types of nucleoside or nucleotide analogs via click chemistry, a strained [3+2] cycloaddition reaction, or a Staudinger ligation.

22. The method of claim 21, wherein the labelling reagent comprises an azide group which binds to nucleoside or nucleotide analogs comprising an alkynyl group via click chemistry.

23. The method of claim 21, wherein the labelling reagent comprises an alkynyl group which binds to nucleoside or nucleotide analogs comprising an azide group via click chemistry.

24. The method of claim 1, wherein the labelling reagent comprises a biotin group.

25. The method of claim 24, wherein the labelling reagent comprising a biotin group is selected from:

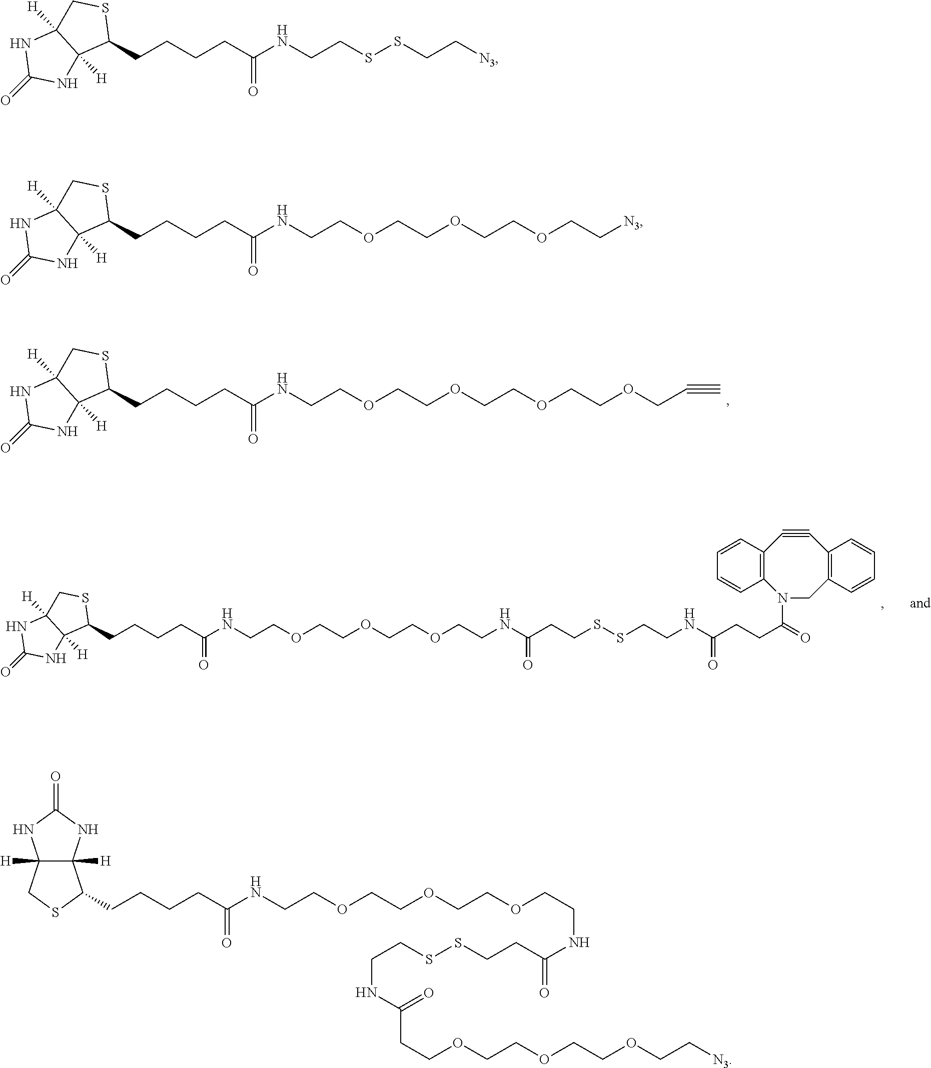 [SMILES: [N-]=[N+]=NCCSSCCNC(=O)CCCC[C@@H]1SC[C@@H]2NC(=O)N[C@@H]21 ; [N-]=[N+]=NCCOCCOCCOCCNC(=O)CCCC[C@@H]1SC[C@@H]2NC(=O)N[C@@H]21 ; C#CCOCCOCCOCCOCCNC(=O)CCCC[C@@H]1SC[C@@H]2NC(=O)N[C@@H]21 ; O=C(CCCC[C@@H]1SC[C@@H]2NC(=O)N[C@@H]21)NCCOCCOCCOCCNC(=O)CCSSCCNC(=O)CCC(=O)N1Cc2ccccc2C#Cc2ccccc21 ; [N-]=[N+]=NCCOCCOCCOCCC(=O)NCCSSCCC(=O)NCCOCCOCCOCCNC(=O)CCCC[C@@H]1SC[C@@H]2NC(=O)N[C@@H]21]

26. The method of claim 1, wherein the labelling reagent further comprises a chemically cleavable linker or enzymatically cleavable linker.

27-29. (canceled)

30. The method of claim 1, wherein a pulldown agent is used to isolate or purified the labelled newly synthesized microbial nucleic acids.

31. The method of claim 30, wherein the pulldown reagent is an antibody immobilized onto a solid support, wherein the antibody binds with high specificity to labelling reagent, or with high specificity to the one or more types of nucleoside or nucleotide analogs.

32. The method of claim 30, wherein the pulldown reagent is streptavidin or avidin immobilized onto a solid support, and wherein the labelling reagent comprises a biotin group.

33. (canceled)

34. The method of claim 1, wherein the labelling reagent or label is removed or cleaved from the isolated or purified newly synthesized microbial nucleic acids prior to (e) of claim 1.

35. The method of claim 1, wherein the identity of the isolated or purified newly synthesized microbial nucleic acids is determined by using a microarray comprising probes to nucleic acids from different microorganisms.

36. The method of claim 35, wherein the identity of the isolated or purified newly synthesized microbial nucleic acids is determined by:
(i) amplifying the isolated or purified newly synthesized microbial nucleic acids using a first PCR based method using primers containing a fluorescent dye to form labelled products, wherein the primers comprise a sequence that is specific to a conserved microbial 16S rRNA gene region;
(ii) applying the labelled products to a microarray comprising probes that comprise unique 16s rRNA variable region sequences from 20 or more microorganisms;
(iii) determining the identity of the viable and/or proliferating microorganisms based upon imaging the microarray for fluorescent hybridization products and determining the identity of the microorganism based upon the sequence of the microarray probe.

37. The method of claim 1, wherein the identity of the isolated or purified newly synthesized microbial nucleic acids is determined or confirmed by sequencing the isolated or purified newly synthesized microbial nucleic acids.

38. The method of claim 37, wherein the isolated or purified newly synthesized microbial nucleic acids are sequenced using a transposome-based sequencing method.

39. The method of claim 38, wherein sequencing of the newly synthesized microbial nucleic acids is by:
(a) applying the isolated or purified newly synthesized microbial nucleic acids to bead-linked transposomes, wherein the bead-linked transposomes mediate the simultaneous fragmentation of microbial nucleic acids and the addition of sequencing primers;
(b) amplifying the microbial nucleic acid fragments with primers that comprise index and adapter sequences to form library of amplified products;
(c) washing and pooling the library of amplified products;
(d) sequencing the library of amplified products; and
(e) determining the identity of the viable and/or proliferating microorganisms based upon correlating the sequences obtained from the library of amplified products with databases of known sequences of microorganisms using bioinformatic analysis.

40. The method of claim 1, wherein the newly synthesized microbial nucleic acids are RNA, wherein the microbial RNA is reversed transcribed into cDNA prior to (e) of claim 1, and wherein the gene expression of the viable and/or proliferating microorganisms can be determined based on analyzing the expression level of gene products from newly synthesized microbial RNA using a microarray and/or by sequencing.

41. A method for determining the effectiveness of an antimicrobial agent in modulating the growth and proliferation of microorganism(s) in a sample, comprising:
(a) obtaining a sample having or suspected of having one or more types of microorganisms;
(b) splitting the sample into two samples, a control sample and a treated sample;
(c) incubating the control sample in the presence of one or more types of nucleoside or nucleotide analogs, wherein the one or more types of nucleoside or nucleotide analogs are incorporated into newly synthesized microbial nucleic acids;
(c′) incubating the treated sample in the presence of one or more types of nucleoside or nucleotide analogs and an antimicrobial agent, wherein the one or more types of nucleoside or nucleotide analogs are incorporated into newly synthesized microbial nucleic acids;
(d) labelling newly synthesized microbial nucleic acids of the control sample and the treated sample by contacting the newly synthesized microbial nucleic acids with a labelling reagent that selectively binds to or with the one or more types of nucleoside or nucleotide analogs;
(e) isolating or purifying the labelled newly synthesized microbial nucleic acids from the control sample and the treated sample;
(f) determining the gene expression level, and/or amounts or identity of the isolated or purified newly synthesized microbial nucleic acids in the control sample;
(f′) determining the gene expression level, and/or amounts and identity of the isolated or purified newly synthesized microbial nucleic acids in the treated sample; and
(g) comparing and determining any changes in the gene expression level and/or amounts and/or identity of the isolated or purified newly synthesized microbial nucleic acids in the control sample with the gene expression level and/or amounts or identity of the isolated or purified newly synthesized microbial nucleic acids in the treated sample,
wherein if there is a decrease in the gene expression level of the newly synthesized microbial nucleic acids in the treated sample v. the control sample, or there is decrease in the amounts and/or identity of the newly synthesized microbial nucleic acids in the treated sample v. the control sample indicates that the antimicrobial agent is effective in modulating the growth and proliferation of the microorganism(s).

42-79. (canceled)
